SEQUENCE LISTING (44 sequences)

SEQ ID NO: 1 - protein, 246 aa, Homo sapiens
MAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 2 - protein, 42 aa, Homo sapiens
MAFLRSMWGVLSALGRSGAELCTGCGSRLRSPFSFVYLPRWF

SEQ ID NO: 3 - protein, 204 aa, Homo sapiens
SSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 4 - protein, 346 aa, Homo sapiens
MAASGKLSTCRLPPLPTIREIIKLLRLQAANELSQNFLLDLRLTDKIVRKAGNLTNAYVYEVGPGPGGITRSILNADVAELLVVEKDTRFIPGLQMLSDAAPGKLRIVHGDVLTFKVEKAFSESLKRPWEDDPPNVHIIGNLPFSVSTPLIIKWLENISCRDGPFVYGRTQMTLTFQKEVAERLAANTGSKQRSRLSVMAQYLCNVRHIFTIPGQAFVPKPEVDVGVVHFTPLIQPKIEQPFKLVEKVVQNVFQFRRKYCHRGLRMLFPEAQRLESTGRLLELADIDPTLRPRQLSISHFKSLCDVYRKMCDEDPQLFAYNFREELKRRKSKNEEKEEDDAENYRL

SEQ ID NO: 5 - protein, 396 aa, Homo sapiens
MWIPVVGLPRRLRLSALAGAGRFCILGSEAATRKHLPARNHCGLSDSSPQLWPEPDFRNPPRKASKASLDFKRYVTDRRLAETLAQIYLGKPSRPPHLLLECNPGPGILTQALLEAGAKVVALESDKTFIPHLESLGKNLDGKLRVIHCDFFKLDPRSGGVIKPPAMSSRGLFKNLGIEAVPWTADIPLKVVGMFPSRGEKRALWKLAYDLYSCTSIYKFGRIEVNMFIGEKEFQKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 6 - DNA, 10 bp, artificial sequence
  synthetic construct - DNA-binding site of SOX9
agaacaatgg

SEQ ID NO: 7 - protein, 11 aa, artificial sequence
  Synthetic polypeptide - a TAT protein transduction domain
YGRKKRRQRRR

SEQ ID NO: 8 - protein, 9 aa, artificial sequence
  synthetic polypeptide - a protein transduction domain
RKKRRQRRR

SEQ ID NO: 9 - protein, 7 aa, artificial sequence
  Synthetic polypeptide - a protein transduction domain
RRRRRRR

SEQ ID NO: 10 - protein, 12 aa, artificial sequence
  synthetic polypeptide - a protein tranduction domain
RRQRRTSKLMKR

SEQ ID NO: 11 - protein, 27 aa, artificial sequence
  synthetic polypeptide - a protein transduction domain
GWTLNSAGYLLGKINLKALAALAKKIL

SEQ ID NO: 12 - protein, 29 aa, artificial sequence
  synethtic polypeptide - a protein transduction domain
WEAKLAKALAKALAKHLAKALAKALKCEA

SEQ ID NO: 13 - protein, 16 aa, artificial sequence
  synthetic polypeptide - a protein transduction domain
RQIKIWFQNRRMKWKK

SEQ ID NO: 14 - protein, 20 aa, artificial sequence
  synthetic polypeptide - an endosomal escape sequence
GDIMGEWGNEIFGAIAGFLG

SEQ ID NO: 15 - protein, 35 aa, artificial sequence
  synthetic polypeptide - a protein transduction domain including an endosomal escape sequence
RRRRRRRRRRRGEGDIMGEWGNEIFGAIAGFLGGE

SEQ ID NO: 16 - protein, 19 aa, artificial sequence
  synthetic polypeptide - predicted sequence from Etfa
MFRAAAPGQLRRAASLLRF

SEQ ID NO: 17 - protein, 35 aa, artificial sequence
  synthetic polypeptide - predicted mitochondrial targeting signal from Dld
MQSWSRVYCSLAKRGHFNRISHGLQGLSAVPLRTY

SEQ ID NO: 18 - protein, 24 aa, artificial sequence
  synthetic polypeptide - a mitochondrial localization signal
MLSRAVCGTSRQLPPVLGYLGSRQ

SEQ ID NO: 19 - protein, 23 aa, artificial sequence
  synthetic polypeptide - a mitochondrial localization signal
LSRAVCGTSRQLPPVLGYLGSRQ

SEQ ID NO: 20 - protein, 24 aa, artificial sequence
  synthetic polypeptide - a mitochondrial localization signal
MLSRAVCGTSRQLAPVLGYLGSRQ

SEQ ID NO: 21 - protein, 23 aa, artificial sequence
  synthetic polypeptide - a mitochondrial localization signal
LSRAVCGTSRQLAPVLGYLGSRQ

SEQ ID NO: 22 - protein, 259 aa, artificial sequence
  synthetic polypeptide - recombinant fusion protein
MARRRRRRRRRRRMAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 23 - protein, 258 aa, artificial sequence
  synthetic polypeptide - recombinant fusion protein
ARRRRRRRRRRRMAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 24 - DNA, 780 bp, artificial sequence
  synthetic polynucleotide - nucleic acid encoding a recombinant fusion protein
atggcgcgtcgtcgtcgtcgtcgtcgtcgtcgtcgtcgtatggcgtttctccgaagcatgtggggcgtgctgagtgccctgggaaggtctggagcagagctgtgcaccggctgtggaagtcgactgcgctcccccttcagttttgtgtatttaccgaggtggttttcatctgtcttggcaagttgtccaaagaaacctgtaagttcttaccttcgattttctaaagaacaactacccatatttaaagctcagaacccagatgcaaaaactacagaactaattagaagaattgcccagcgttggagggaacttcctgattcaaagaaaaaaatatatcaagatgcttatagggcggagtggcaggtatataaagaagagataagcagatttaaagaacagctaactccaagtcagattatgtctttggaaaaagaaatcatggacaaacatttaaaaaggaaagctatgacaaaaaaaaaagagttaacactgcttggaaaaccaaaaagacctcgttcagcttataacgtttatgtagctgaaagattccaagaagctaagggtgattcaccgcaggaaaagctgaagactgtaaaggaaaactggaaaaatctgtctgactctgaaaaggaattatatattcagcatgctaaagaggacgaaactcgttatcataatgaaatgaagtcttgggaagaacaaatgattgaagttggacgaaaggatcttctacgtcgcacaataaagaaacaacgaaaatatggtgctgaggagtgttaa

SEQ ID NO: 25 - protein, 264 aa, artificial sequence
  synthetic polypeptide - recombinant fusion protein
MRRRRRRRRRRRGEGDIMGEWGNEIFGAIAGFLGGEMLSRAVCGTSRQLPPVLGYLGSRQSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 26 - protein, 263 aa, artificial sequence
  synthetic polypeptide - recombinant fusion protein
RRRRRRRRRRRGEGDIMGEWGNEIFGAIAGFLGGEMLSRAVCGTSRQLPPVLGYLGSRQSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 27 - DNA, 795 bp, artificial sequence
  synthetic polypeptide - nucleic acid encoding a recombinant fusion protein
atgcggcgacgcagacgtcgtcgtcggcggcgtcgcggcgagggtgatattatgggtgaatgggggaacgaaattttcggagcgatcgctggttttctcggtggagaaatgttatcacgcgcggtatgtggcaccagcaggcagctgcctccagtccttggctatctgggttcccgccagtcatcggtgttagcatcatgtccgaaaaaacctgtctcgtcgtacctgcgcttctccaaagagcagctgccgatttttaaagcgcaaaatccggatgctaaaacgactgaactgattcgccgcattgcacaacgctggcgcgaactcccggacagtaaaaaaaaaatttatcaggacgcctatcgggctgaatggcaggtctataaagaggagatctcacgcttcaaagaacaattaaccccgagtcaaataatgtctctggaaaaagaaatcatggataaacacttaaaacgaaaggcgatgacgaagaaaaaagaactgaccctgctaggtaaacctaagcgtccgcgctctgcgtataatgtgtacgtggcagaacgttttcaggaggccaaaggggattctccgcaagaaaaactgaagaccgtcaaagaaaattggaaaaacctgtctgatagcgaaaaagaactgtacattcagcacgctaaagaagatgagacgcggtatcacaacgaaatgaaatcttgggaagagcagatgatcgaggtcggtcggaaggatcttctccgtcgaaccatcaaaaaacagcgtaaatatggagcagaagagtgctga

SEQ ID NO: 28 - protein, 7 aa, artificial sequence
  synthetic polypeptide - a nuclear localization signal
GKKRSKV

SEQ ID NO: 29 - protein, 7 aa, artificial sequence
  synthetic polypeptide - a nuclear localization signal
KSRKRKL

SEQ ID NO: 30 - protein, 19 aa, artificial sequence
  synthetic polypeptide - a nuclear localization signal
KRPAATKKAGQAKKKKLDK

SEQ ID NO: 31 - protein, 20 aa, artificial sequence
  synthetic polypeptide - a nuclear localization signal
RKKRKTEEESPLKDKAKKSK

SEQ ID NO: 32 - protein, 20 aa, artificial sequence
  synthetic polypeptide - a nuclear localization signal
KDCVMNKHHRNRCQYCRLQR

SEQ ID NO: 33 - protein, 9 aa, artificial sequence
  synthetic polypeptide - a nuclear localization signal
PAAKRVKLD

SEQ ID NO: 34 - protein, 20 aa, artificial sequence
  synthetic polypeptide - a nuclear localization signal
KKYENVVIKRSPRKRGRPRK

SEQ ID NO: 35 - protein, 8 aa, artificial sequence
  synthetic polypeptide - protein purification tag
DYKDDDDK

SEQ ID NO: 36 - protein, 6 aa, artificial sequence
  synthetic polypeptide - protein purification tag
YPYDVP

SEQ ID NO: 37 - protein, 10 aa, artificial sequence
  synthetic polypeptide - protein purification tag
ILKKATAYIL

SEQ ID NO: 38 - protein, 10 aa, artificial sequence
  synthetic polypeptide - protein purification tag
EQKLISEEDL

SEQ ID NO: 39 - protein, 1230 aa, Homo sapiens
MSALCWGRGAAGLKRALRPCGRPGLPGKEGTAGGVCGPRRSSSASPQEQDQDRRKDWGHVELLEVLQARVRQLQAESVSEVVVNRVDVARLPECGSGDGSLQPPRKVQMGAKDATPVPCGRWAKILEKDKRTQQMRMQRLKAKLQMPFQSGEFKALTRRLQVEPRLLSKQMAGCLEDCTRQAPESPWEEQLARLLQEAPGKLSLDVEQAPSGQHSQAQLSGQQQRLLAFFKCCLLTDQLPLAHHLLVVHHGQRQKRKLLTLDMYNAVMLGWARQGAFKELVYVLFMVKDAGLTPDLLSYAAALQCMGRQDQDAGTIERCLEQMSQEGLKLQALFTAVLLSEEDRATVLKAVHKVKPTFSLPPQLPPPVNTSKLLRDVYAKDGRVSYPKLHLPLKTLQCLFEKQLHMELASRVCVVSVEKPTLPSKEVKHARKTLKTLRDQWEKALCRALRETKNRLEREVYEGRFSLYPFLCLLDEREVVRMLLQVLQALPAQGESFTTLARELSARTFSRHVVQRQRVSGQVQALQNHYRKYLCLLASDAEVPEPCLPRQYWEELGAPEALREQPWPLPVQMELGKLLAEMLVQATQMPCSLDKPHRSSRLVPVLYHVYSFRNVQQIGILKPHPAYVQLLEKAAEPTLTFEAVDVPMLCPPLPWTSPHSGAFLLSPTKLMRTVEGATQHQELLETCPPTALHGALDALTQLGNCAWRVNGRVLDLVLQLFQAKGCPQLGVPAPPSEAPQPPEAHLPHSAAPARKAELRRELAHCQKVAREMHSLRAEALYRLSLAQHLRDRVFWLPHNMDFRGRTYPCPPHFNHLGSDVARALLEFAQGRPLGPHGLDWLKIHLVNLTGLKKREPLRKRLAFAEEVMDDILDSADQPLTGRKWWMGAEEPWQTLACCMEVANAVRASDPAAYVSHLPVHQDGSCNGLQHYAALGRDSVGAASVNLEPSDVPQDVYSGVAAQVEVFRRQDAQRGMRVAQVLEGFITRKVVKQTVMTVVYGVTRYGGRLQIEKRLRELSDFPQEFVWEASHYLVRQVFKSLQEMFSGTRAIQHWLTESARLISHMGSVVEWVTPLGVPVIQPYRLDSKVKQIGGGIQSITYTHNGDISRKPNTRKQKNGFPPNFIHSLDSSHMMLTALHCYRKGLTFVSVHDCYWTHAADVSVMNQVCREQFVRLHSEPILQDLSRFLVKRFCSEPQKILEASQLKETLQAVPKPGAFDLEQVKRSTYFFS

SEQ ID NO: 40 - protein, 7 aa, artificial sequence
  synthetic polypeptide - a nuclear localization signal
PKKKRKV

SEQ ID NO: 41 - protein, 88 aa, artificial sequence
  synthetic polypeptide - antibody binding domain
HDEAQQNAFYQVLNMPNLNADQRNGFIQSLKDDPSQSANVLGEAHDEAQQNAFYQVLNMPNLNADQRNGFIQSLKDDPSQSANVLGEA

SEQ ID NO: 42 - protein, 91 aa, artificial sequence
  synthetic polypeptide - antibody binding domain
HDEAQQNAFYQVLNMPNLNADQRNGFIQSLKDDPSQSANVLGEAHDEAQQNAFYQVLNMPNLNADQRNGFIQSLKDDPSQSANVLGEAGEG

SEQ ID NO: 43 - protein, 331 aa, artificial sequence
  synthetic polypeptide - recombinant fusion protein
MRRRRRRRRRRRGEGDIMGEWGNEIFGAIAGFLGGEHDEAQQNAFYQVLNMPNLNADQRNGFIQSLKDDPSQSANVLGEAHDEAQQNAFYQVLNMPNLNADQRNGFIQSLKDDPSQSANVLGEAGEGSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 44 - protein, 330 aa, artificial sequence
  synthetic polypeptide - recombinant fusion protein
RRRRRRRRRRRGEGDIMGEWGNEIFGAIAGFLGGEHDEAQQNAFYQVLNMPNLNADQRNGFIQSLKDDPSQSANVLGEAHDEAQQNAFYQVLNMPNLNADQRNGFIQSLKDDPSQSANVLGEAGEGSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

(19) United States
(12) Patent Application Publication
(10) Pub. No.: US 2015/0283233 A1
(43) Pub. Date: Oct. 8, 2015

(54) Compositions and Methods for Enhancing Immune Responses

(71) Applicant: Gencia Corporation, Charlottesville, VA (US)

(72) Inventor: Shaharyar Khan, Charlottesville, VA (US)

(21) Appl. No.: 14/408,159

(22) PCT Filed: Jun. 17, 2013
(86) PCT No.: PCT/US2013/046132

§ 371 Date: Dec. 15, 2014

Related U.S. Application Data
(60) Provisional application No. 61/660,223, filed on Jun. 15, 2012.

(51) Int. Cl. A61K 39/39; A61K 39/00

(52) U.S. Cl. CPC A61K 39/39; A61K 39/0011; A61K 2039/55516

(57) ABSTRACT

Compositions and methods for inducing, enhancing, or promoting an immune response are disclosed. In some embodiments the increased immune response is against an antigen such as a tumor antigen or a viral antigen. Immune responses include activation of cytotoxic T cells to kill cells displaying the antigen. In some embodiments, the methods include contacting cells with an effective amount of a transcription factor A-mitochondrial (TFAM) fusion protein to increase expression of a Major Histocompatibility Complex I (MHC I), reduce expression of HLA-G on the surface of cells, or increase expression of one or more cytokines or chemokines Methods of treating cancer are also disclosed.

### FIELD OF THE INVENTION

The field of the invention generally relates to compositions and methods of enhancing an immune response in a patient in need thereof.

### BACKGROUND OF THE INVENTION

The body's natural defense to pathogens and cancer include responses orchestrated by the immune system. For example, the major histocompatibility complex (MHC) class I molecules are one of two primary classes of MHC molecules and are found on every nucleated cell of the body. MHC I molecules display peptides on the cell's surface where the peptides can be detected by patrolling immune cells Immune cells attack cells displaying proteins that are not commonly expressed by normal cells, such as those from cancer cells or proteins originating from pathogens such as viruses or intracellular microorganisms.

Pathogens and tumors have evolved mechanisms to evade the immune surveillance system by altering the expression of antigen presenting molecules such as MHC I, or by altering the expression of immune receptors that recognize antigen presenting molecules. For example, tumor cells can increase expression of immune suppressing cell surface receptors such as human leukocyte antigen G (HLA-G), a non-classical MHC I molecule.

Numerous immunotherapies have been developed in an attempt to increase or direct the body's immune response to pathogens or cancer, however, many of these immunotherapies fail. Therefore, there remains a need for immunotherapies that increase the body's natural response to foreign antigens.

It is an object of the invention to provide compositions and methods for increasing an immune response in a subject.

It is another object of the invention to provide compositions and methods for increasing expression of MHC I molecules.

It is a further object of the invention to provide compositions and methods for increasing express of MHC I molecules on specific cells, for example cancer cells.

It is another object of the invention to provide compositions and methods for decreasing expression of HLA-G molecules on specific cells for example cancer cells.

It is another object of the invention to increase the level of pro-inflammatory cytokines in and around tumors.

It is yet another object of the invention to provide compositions and methods for enhancing or improving immunotherapies.

### SUMMARY OF THE INVENTION

Compositions and methods for inducing, enhancing, or promoting an immune response against an antigen are disclosed. The methods include contacting cells with an effective amount of a transcription factor A-mitochondrial (TFAM) or a fusion protein thereof to increase expression of a MHC I, reduce expression of HLA-G on the surface of cells, increase expression of one or more cytokines or chemokines, or a combination thereof. Immune responses include activation of cytotoxic T cells to kill cells displaying an antigen not recognized as self. In preferred embodiments, the antigen is a tumor antigen.

Also disclosed are methods of treating cancer. Some embodiments include administering to a patient in need thereof an effective amount of a transcription factor A-mitochondrial (TFAM) fusion protein to induce, enhance, or promote an immune response against cancer cells. TFAM fusion protein can (1) increase expression of HLA-A, HLA-B, or HLA-C on the surface of cancer cells, (2) decrease expression of HLA-G on the surface of cancer cells, (3) increase expression of one or more cells in or around the microenvironment of the cancer cells, or (4) combinations thereof. In some embodiment, the TFAM fusion protein is co-administered in combination with a second therapeutic, such as a second immunotherapeutic.

### BRIEF DESCRIPTION OF THE DRAWINGS

FIG. 1 is a bar graph showing the presence of MHC I molecules on isolated from nude mouse pancreatic carcinoma cell (Mia PaCa 2) tumor xenographs, following treatment of the mouse with vehicle, or low (0.33 mg/kg), mid1 (0.5 mg/kg) or mid2 (0.66 mg/kg) dose of rhTFAM. The graph is normalized to vehicle control (100%). N=4, average values and median deviation shown.

FIG. 2 is a bar graph showing the presence of MHC I molecules on cells isolated from nude mouse Glioblastoma Multiforme (GBM) tumor xenographs, following treatment of the mouse with vehicle, or low (0.33 mg/kg), mid1 (0.5 mg/kg), mid2 (0.66 mg/kg), or high (1.0 mg/kg) dose of rhTFAM. The graph is normalized to vehicle control (100%). N=4, average values and median deviation shown.

FIG. 3 is a bar graph showing the presence of HLA-G molecules on cells isolated from nude mouse MCF-7 tumor xenographs, following treatment of the mouse with rhTFAM-50 (0.33 mg/kg in 50 n1 injection volume), rhTFAM-75 (0.5 mg/kg in 75 μl injection volume), rhTFAM-100 (0.66 mg/kg in 100 μl injection volume), or rhTFAM-150 (1.0 mg/kg in 150 μl injection volume) dose of rhTFAM. The graph is normalized to rhTFAM-50 (100%).

FIG. 4 is a bar graph showing CXCL1 levels in pancreatic carcinoma cell (Mia PaCa 2) tumor xenographs, following treatment of the mouse with vehicle control, or low (0.33 mg/kg), mid1 (0.5 mg/kg), mid2 (0.66 mg/kg), or high (1.0 mg/kg) dose of rhTFAM.

FIG. 5 is a bar graph showing MIP-1α levels in pancreatic carcinoma cell (Mia PaCa 2) tumor xenographs, following treatment of the mouse with vehicle control, or low (0.33 mg/kg), mid1 (0.5 mg/kg), mid2 (0.66 mg/kg), or high (1.0 mg/kg) dose of rhTFAM.

FIG. 6 is a bar graph showing MIP-1β levels in pancreatic carcinoma cell (Mia PaCa 2) tumor xenographs, following treatment of the mouse with vehicle control, or low (0.33 mg/kg), mid1 (0.5 mg/kg), mid2 (0.66 mg/kg), or high (1.0 mg/kg) dose of rhTFAM.

FIG. 7 is a bar graph showing RANTES levels in pancreatic carcinoma cell (Mia PaCa 2) tumor xenographs, following treatment of the mouse with vehicle control, or low (0.33 mg/kg), mid1 (0.5 mg/kg), mid2 (0.66 mg/kg), or high (1.0 mg/kg) dose of rhTFAM.

FIG. 8 is a bar graph showing IL-1β levels in pancreatic carcinoma cell (Mia PaCa 2) tumor xenographs, following treatment of the mouse with vehicle control, or low (0.33 mg/kg), mid1 (0.5 mg/kg), mid2 (0.66 mg/kg), or high (1.0 mg/kg) dose of rhTFAM.

FIG. 9 is a bar graph showing TNF-α levels in pancreatic carcinoma cell (Mia PaCa 2) tumor xenographs, following treatment of the mouse with vehicle control, or low (0.33 mg/kg), mid1 (0.5 mg/kg), mid2 (0.66 mg/kg), or high (1.0 mg/kg) dose of rhTFAM.

FIG. 10 is a photo of autoradiograph of a western blot detecting MHC I, or beta-actin (loading control) in protein isolated from LN229 brain tumor xenographs from mice treated with a series vehicle (PBS) or 10 μg of rhTFAM every fourth day by tail vain injection.

FIG. 11 is a photo of autoradiograph of a western blot detecting MHC I, or beta-actin (loading control) in protein isolated from LN229 brain tumor xenographs from mice treated with a series vehicle (PBS), 5 μg of rhTFAM, 40 mg/kg Gemcitabin, or 5 μg of rhTFAM and 40 mg/kg Gemcitabin every fourth day by tail vain injection.

### DETAILED DESCRIPTION OF THE INVENTION

### I. Definitions

In describing and claiming the disclosed subject matter, the following terminology will be used in accordance with the definitions set forth below. The terms “individual”, “host”, “subject”, and “patient” are used interchangeably herein, and refer to a mammal, including, but not limited to, humans, rodents, such as mice and rats, and other laboratory animals.

The term “drug” refers to small molecules, protein therapeutics, vaccines, and immunomodulators.

As used herein, the term “treating” generally includes increasing, enhancing, or promoting an immune response.

The term “reduce”, “inhibit”, “alleviate,” “decrease,” “increase,” “enhance,” and “promote,” are typically used relative to a control. One of skill in the art would readily identify the appropriate control to use for each experiment. For example a decreased response in a subject or cell treated with a compound is compared to a response in subject or cell that is not treated with the compound.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “low stringency” refers to conditions that permit a polynucleotide or polypeptide to bind to another substance with little or no sequence specificity.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein the term “isolated” is meant to describe a compound of interest (e.g., nucleic acids, polypeptides, etc.) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids or polypeptides are at least 60% free, preferably 75% free, and most preferably 90% free from other associated components.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

“Localization Signal or Sequence or Domain” or “Targeting Signal or Sequence or Domain” are used interchangeably and refer to a signal that directs a molecule to a specific cell, tissue, organelle, intracellular region or cell state. The signal can be polynucleotide, polypeptide, or carbohydrate moiety or can be an organic or inorganic compound sufficient to direct an attached molecule to a desired location. Exemplary targeting signals include mitochondrial localization signals from the precursor proteins list in U.S. Pat. No. 8,039,587, and cell targeting signals known in the art such as those in Wagner et al., Adv Gen, 53:333-354 (2005) the disclosures of which are specifically incorporated by reference herein in their entireties. It will be appreciated that the entire sequence need not be included, and modifications including truncations of these sequences are within the scope of the disclosure provided the sequences operate to direct a linked molecule to a specific cell type. Targeting signals of the present disclosure can have 80 to 100% sequence identity to the mitochondrial localize signal or cell targeting signal sequences. One class of suitable targeting signals include those that do not interact with the targeted cell in a receptor:ligand mechanism. For example, targeting signals include signals having or conferring a net charge, for example a positive charge. Positively charged signals can be used to target negatively charged cell types such as neurons and muscle. Negatively charged signals can be used to target positively charged cells.

“Tropism” refers to the propensity of a molecule to be attracted to a specific cell, cell type or cell state. In the art, tropism can refer to the way in which different viruses and pathogens have evolved to preferentially target to specific host species, or specific cell types within those species. The propensity for a molecule to be attracted to a specific cell, cell type or cell state can be accomplished by means of a targeting signal.

“Cell Type” is a manner of grouping or classifying cells in the art. The term cell type refers to the grouping of cells based on their biological character determined in part through common biological function, location, morphology, structure, expression of polypeptides, nucleotides or metabolites.

“Cell State” refers to the condition of a cell type. Cells are dynamic throughout their life and can achieve various states of differentiation, function, morphology and structure. As used herein, cell state refers to a specific cell type throughout its lifetime.

“Cell surface marker” refers to any molecule such as moiety, peptide, protein, carbohydrate, nucleic acid, antibody, antigen, and/or metabolite presented on the surface or in the vicinity of a cell sufficient to identify the cell as unique in either type or state.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “low stringency” refers to conditions that permit a polynucleotide or polypeptide to bind to another substance with little or no sequence specificity.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

“Localization Signal or Sequence or Domain” or “Targeting Signal or Sequence or Domain” are used interchangeably and refer to a signal that directs a molecule to a specific cell, tissue, organelle, intracellular region or cell state. The signal can be polynucleotide, polypeptide, or carbohydrate moiety or can be an organic or inorganic compound sufficient to direct an attached molecule to a desired location. Exemplary targeting signals include mitochondrial localization signals from the precursor proteins list in U.S. Pat. No. 8,039,587, and cell targeting signals known in the art such as those in Wagner et al., Adv Gen, 53:333-354 (2005) the disclosures of which are specifically incorporated by reference herein in their entireties. It will be appreciated that the entire sequence need not be included, and modifications including truncations of these sequences are within the scope of the disclosure provided the sequences operate to direct a linked molecule to a specific cell type. Targeting signals of the present disclosure can have 80 to 100% sequence identity to the mitochondrial localize signal or cell targeting signal sequences. One class of suitable targeting signals include those that do not interact with the targeted cell in a receptor:ligand mechanism. For example, targeting signals include signals having or conferring a net charge, for example a positive charge. Positively charged signals can be used to target negatively charged cell types such as neurons and muscle. Negatively charged signals can be used to target positively charged cells.

“Tropism” refers to the propensity of a molecule to be attracted to a specific cell, cell type or cell state. In the art, tropism can refer to the way in which different viruses and pathogens have evolved to preferentially target to specific host species, or specific cell types within those species. The propensity for a molecule to be attracted to a specific cell, cell type or cell state can be accomplished by means of a targeting signal.

“Cell Type” is a manner of grouping or classifying cells in the art. The term cell type refers to the grouping of cells based on their biological character determined in part through common biological function, location, morphology, structure, expression of polypeptides, nucleotides or metabolites.

“Cell State” refers to the condition of a cell type. Cells are dynamic throughout their life and can achieve various states of differentiation, function, morphology and structure. As used herein, cell state refers to a specific cell type throughout its lifetime.

“Cell surface marker” refers to any molecule such as moiety, peptide, protein, carbohydrate, nucleic acid, antibody, antigen, and/or metabolite presented on the surface or in the vicinity of a cell sufficient to identify the cell as unique in either type or state.

### II. Methods

A. Methods for Modulating an Immune Response

The compositions disclosed herein are which are useful in methods for modulating an immune response in a subject. As used herein, the terms “immunologic”, “immunological” or “immune” response is the development of a beneficial humoral (antibody mediated) and/or a cellular (mediated by antigen-specific T cells or their secretion products) response in a patient. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject. In some embodiments, the immune response is measured by reduced tumor burden or pathogen burden. The disclosed compositions can act on the immune cells, the immune target cells, or combinations thereof to elicit the increased immune response. Examples of immune target cells include, but are not limited to, cancer cells, tumor cells, infected cells, such as cells infected with virus.

The methods typically include administering a subject with one or more of the disclosed compositions discussed in more detail below. The methods can be used for the treatment of cancer or infectious disease (i.e. viral infections such as HIV). As discussed in more detail below, in some embodiments, the compositions can modulate effector cells or molecules to target and destroy cancer cells or virus infected cells. For example, effector cells can be cytotoxic T cells and effector molecules can be monoclonal antibodies. The effector cells and molecules can provide therapeutic benefit either directly or indirectly by activating cytotoxic T cells or preventing inactivation of cytotoxic T cells.

The compositions can be prophylactic or therapeutic. Prophylactic methods are aimed to prevent disease or infection. Therapeutic methods are used to treat the disease or infection.

Suitable compositions for use with the disclosed methods are discussed in detail below and typically include an effective amount of a mitochondrial DNA-binding polypeptide, preferably a mitochondrial transcription factor or polynucleotide-binding fragment thereof. Examples mitochondrial DNA-binding polypeptides include, but are not limited to, mitochondrial transcription factors such as transcription factor A, mitochondrial (TFAM), transcription factor B1, mitochondrial (TFB1M), transcription factor B2, mitochondrial (TFB2M), Polymerase (RNA) Mitochondrial (DNA directed) (POLRMT); and functional fragments, variants, and fusion polypeptides thereof.

Exemplary fusion proteins containing a mitochondrial transcription factor polypeptide are disclosed in U.S. Pat. Nos. 8,039,587, 8,062,891, 8,133,733, and U.S. Published Application Nos. 2009/0123468, 2009/0208478, and 2006/0211647 all of which are specifically incorporated by reference herein their entireties.

In preferred embodiments the composition includes a mitochondrial DNA-binding polypeptide. The mitochondrial DNA-binding polypeptide can be a recombinant fusion protein including a mitochondrial DNA-binding polypeptide, a protein transduction domain, and optionally one or more targeting signals. In some embodiments, the disclosed compositions cause an increase in mitochondrial number, an increase in mitochondrial respiration, an increase mitochondrial Electron Transport Chain (ETC) activity, increased oxidative phosphorylation, increased oxygen consumption, increased ATP production, or combinations thereof relative to a control. In preferred embodiments, the disclosed methods cause a reduction in oxidative stress in the subject compared to a control.

1. Increasing Expression of MHC Molecules

In some embodiments, the compositions disclosed herein are administered to a subject in an effective amount to induce, increase, enhance or promote the expression of an MHC molecule on the surface of cells of the subject. The immune system uses a class of molecules called the major histocompatability complex (MHC) to identify cells as self or non-self. The MHC can be divided into two main groups: class I (MHC I) and class II (MHC II).

Major Histocompatability Complex I (MHC I) is a heterodimer transmembrane ligand, expressed on all nucleated cells and can be complexed with a peptide derived from an intracellular protein. MHC I molecules typically present peptides derived from intracellular proteins. The intracellular proteins include both self and non-self proteins. Cytotoxic T lymphocytes (CTL) express the cell surface receptor for MHC I, namely the T cell receptor (TCR). The TCR identifies the peptides presented by, or complexed with, MHC I as originating from either the cell itself (self) or as originating from a pathogen that has invaded the cell such as a virus. Cancer cells can exhibit deregulated gene expression, leading to deregulated expression of proteins or splice isoforms, as well as abnormal expression of non-native fusion proteins. As such, these proteins may be recognized as “non-self” by immune surveillance cells. A normal cell displays peptides from normal cellular protein turnover on its class I MHC, and cytotoxic T cells (CTLs) are not activated in response to them due to central and peripheral tolerance mechanisms. When a cell expresses foreign proteins, or proteins not normally expressed in an immune-surveyed tissue, such as viral peptides or tumor antigens, a fraction of the MHC I will display these peptides on the cell surface as MHC I:peptide complex. CTLs specific for the MHC I:peptide complex will recognize and kill the presenting cell.

A defense mechanism for virus infected and cancer cells is the down-regulation of MHC I molecules in order to avoid recognition by immune cells.

Increasing MHC I molecules on the surface of cells can greatly increase the body's natural immune response to find and kill the cancer cell. In some embodiments, the compositions disclosed herein are administered in an effective amount to induce, increase, enhance or promote expression of an MHC I:peptide complex on the surface of the cells of the subject. The MHC I molecules can be divided into MHC Ia and MHC Ib, or classical and non-classical, respectively. Examples of MHC Ia molecules are human leukocyte antigens (HLA) A, B and C. In some embodiments, the compositions are administered to a subject in an amount effective to induce, increase, enhance or promote cell surface expression of MHC I:peptide complex having classical class I heavy chain. Therefore, in some embodiments, the compositions are administered to a subject in an amount effective to cell surface expression of MHC I:peptide complexes having HLA-A, HLA-B, HLA-C, or combinations thereof.

Preferably, the peptide of the peptide complex is an immunogenic peptide such as peptides derived from viral proteins or tumor proteins. In some embodiments increased cell surface expression of MHC I:peptide complex induces, increases, enhances or promotes of an immune response against a virus or tumor antigen. For example, in some embodiments an increase in MHC I:peptide complex on the surface of cells mediates an increase in the number of cytotoxic T cells activated against the peptide. Therefore, in some embodiments, the disclosed compositions are administered in an effective amount increase the number of activated CTLs in a subject.

In some embodiments, the compositions are targeted to cells of interest, such as virus-infected or cancer cells.

In some embodiments, the compositions disclosed herein as used to reduce a decrease in MHC I expression found in patient treated with a chemotherapeutic agent. Example IV and FIG. 11 below shows that chemotherapeutic drugs such as gemcitabine can induce a decrease in the expression of MHC I. The decrease in MHC I following administration of chemotherapy can be reduced by the disclosed compositions. Therefore, in some embodiments, the disclosed compositions are administered in an effective amount to prevent, reduce, inhibit or alleviate the decrease of MHC I expression associated with administration with a chemotherapeutic agent such as gemcitabine.

2. HLA-G

In some embodiments, the compositions disclosed herein are administered to a subject in an effective amount to reduce, decrease, or inhibit the expression of HLA-G on the surface of cells of the subject. HLA-G is a non-classical class Ib HLA molecule that differs from other classical class I HLA (-A, -B and -C) molecules in a number of ways. For example, HLA-G has (1) limited protein variability, (2) presence of several membrane-bound and soluble isoforms, generated by alternative splicing of the primary transcript, (3) unique molecular structure, presenting a reduced cytoplasmic tail, (4) modulation of the immune response, and (5) restricted tissue expression (Donadi, et al., Cell Mol Life Sci. 68(3): 369-395 (2011)).

HLA-G is believed to be a tolerogenic molecule. HLA-G in known to play a role in pregnancy as it is commonly expressed on trophoblast cells in the uterus (Rizzo et al, Cell Mol Life Sci 68:341-52 (2011)). Other lines of evidence suggest the molecule also plays an important role in the suppression of the immune response (Carosella, et al., Blood, 111:4862-4870 (2008), Carosella, et al., Trends Immunol., 29:125-132 (2008)). HLA-G1 inhibits the cytolytic function of uterine and peripheral blood NK cells, the antigen-specific cytolytic function of cytotoxic T lymphocytes, the alloproliferative response of CD4+ T cells, the proliferation of T cells and peripheral blood NK cells, and the maturation and function of dendritic cells. For example, HLA-G can inhibit immunocompetent cells upon binding to inhibitory receptors expressed by the immunocompetent cells. Inhibitory receptors include but are not limited to immunoglobulin-like transcript (ILT)-2, ILT-4 and KIR2DL4/p49. These receptors can be found on lymphoid cells, dendritic cells, macrophages, monocytes and natural killer (NK) cells (Rouas-Freiss et al., Cancer Res, 65:10139-10144 (2005)).

It has been discovered that the compositions disclosed herein can reduce the expression of HLA-G on the surface of cells. Therefore, in some embodiments the disclosed compositions induce an immune response by reducing, decreasing, or inhibiting a toleragenic signal. For example, in some embodiments a decrease in HLA-G promotes the cytolytic function of uterine and peripheral blood NK cells, the antigen-specific cytolytic function of cytotoxic T lymphocytes, the alloproliferative response of CD4+ T cells, the proliferation of T cells and peripheral blood NK cells, and the maturation and function of dendritic cells. Therefore, in some embodiments, the disclosed compositions are administered in an amount effective to promote the cytolytic function of uterine and peripheral blood NK cells, the antigen-specific cytolytic function of cytotoxic T lymphocytes, the alloproliferative response of CD4+ T cells, the proliferation of T cells and peripheral blood NK cells, the maturation of dendritic cells, the function of dendritic cells, or combinations thereof in a subject

HLA-G is found on cancer cells and may help cancer cells by inhibiting immunocompetent cells that might otherwise kill the cancer cell (Amiot, et al., Cell Mol Life Sci 68:417-31 (2011)). In preferred embodiments, the disclosed compositions are administered in an effective amount to decrease HLA-G expression on a cancer cell. In some embodiments, the composition is specifically targeted to the cancer cells.

3. Cytokine and Chemokine Expression

In some embodiments, the compositions disclosed herein are administered to a subject in an effective amount to induce, increase, enhance or promote the expression of one or more pro-inflammatory cytokines or chemokines. Cytokines are typically small cell-signaling protein molecules, such as interleukins and interferons that function in intercellular communication. Chemokines are typically small cell signaling protein molecules that induce chemotaxis. Both cytokines and chemokines have important roles in modulating immune response. In some embodiments, the disclosed compositions are administered in an effective amount to increase or induce a cytokine or chemokine mediated immune response against a virus or tumor antigen. When treating cancer, cytokine or chemokine expression can be increased in or around a tumor microenvironment. The cytokine or chemokine can be a lymphocyte expressed cytokine or chemokine, or macrophage expressed cytokine or chemokine. Cytokine or chemokines include, but are not limited to CXCL1, MIP-1α, MIP-1μ, RANTES, IL-1β, TNF-α, or combinations thereof.

B. Methods of Treatment

1. Diseases to be Treated

It is possible to treat both a disease and symptoms of a disease. Treating a disease can involve providing whatever the deficiency may be that is the cause of the disease. For example, a disease caused by a mutation that results in a non-functional protein can be treated by providing a functional version of that protein. Treating the symptoms of disease refers to any treatment that stabilizes or decreases at least one symptom of the disease. In other words, treating symptoms means that the treatment targets a result of the disease, not the disease itself.

a. Cancer

Any type of cancer can be prevented, treated and/or managed in accordance with the disclosed methods and compositions. Non-limiting examples of cancers that can be prevented, treated and/or managed include: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

b. Infectious Disease

The compositions and methods disclosed herein are also useful for treatment infectious diseases, particularly viral infection. The following are examples of infectious diseases which can be treated by the methods and compositions disclosed herein: Viral—AIDS, AIDS Related Complex, Chickenpox (Varicella), Common cold, Cytomegalovirus Infection, Colorado tick fever, Dengue fever, Ebola haemorrhagic fever, Epidemic parotitis, Flu, Hand, foot and mouth disease, Hepatitis—Herpes simplex, Herpes zoster, HPV, Influenza, Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease—Yellow fever; Bacterial—Anthrax, Bacterial Meningitis, Brucellosis, Bubonic plague, Campylobacteriosis, Cat Scratch Disease, Cholera, Diphtheria, Epidemic Typhus, Gonorrhea, Hansen's Disease, Legionellosis, Leprosy, Leptospirosis, Listeriosis, Lyme Disease, Melioidosis, MRSA infection, Nocardiosis, Pertussis, Pneumococcal pneumonia, Psittacosis, Q fever, Rocky Mountain Spotted Fever or RMSF, Salmonellosis, Scarlet Fever, Shigellosis, Syphilis, Tetanus, Trachoma, Tuberculosis, Tularemia, Typhoid Fever, Typhus, Whooping Cough; Parasitic—African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, Trypanosomiasis, Pneumonia, Meningitis, Sepsis, Peritonitis, Arthritis (Infectious), Abscess, Carbuncle.

2. Methods of Administration

The compositions provided herein may be administered in a physiologically acceptable carrier to a host. Preferred methods of administration include systemic or direct administration to a cell. The compositions can be administered to a cell or subject, as is generally known in the art for gene therapy applications. In gene therapy applications, the compositions are introduced into cells in order to transfect an organelle. “Gene therapy” includes both conventional gene therapy where a lasting effect is achieved by a single treatment, and the administration of gene therapeutic agents, which involves the one time or repeated administration of a therapeutically effective DNA or RNA.

The modified complex compositions can be combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

a. Parental Administration

The compositions of the present disclosure can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, reconsitutable dry (i.e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1,3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutically acceptable polymeric or hydrophobic materials such as emulsions, ion exchange resins, sparingly soluble polymers, and sparingly soluble salts.

b. Buccal Formulations

Pharmaceutical compositions may be prepared, packaged, or sold in a buccal formulation. Such formulations may be in the form of tablets, powders, aerosols, atomized solutions, suspensions, or lozenges made using known methods, and may contain from about 0.1% to about 20% (w/w) active ingredient with the balance of the formulation containing an orally dissolvable or degradable composition and/or one or more additional ingredients as described herein. Preferably, powdered or aerosolized formulations have an average particle or droplet size ranging from about 0.1 nanometers to about 200 nanometers when dispersed.

c. Additional Ingredients

As used herein, “additional ingredients” include one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known. Suitable additional ingredients are described in Remington's Pharmaceutical Sciences, Mack Publishing Co., Genaro, ed., Easton, Pa. (1985).

d. Dosages

Dosages and desired concentrations of the pharmaceutical compositions of the present disclosure may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

The amount or dose of the material administered should be sufficient to affect a therapeutic or prophylactic response in a subject over a reasonable time frame. For example, the dose of the material should be sufficient to increase the amount of MHC I on the cell surface. The dose should be sufficient to stimulate tumor cell killing, stimulate CD8+ T cells, and/or treat or prevent cancer in a period of from about 2 hours or longer from the time of administration.

Many assays for determining an administered dose are known in the art. For purposes of the present methods, an assay, which comprises comparing the extent to which CD8+ T cells are stimulated to kill tumor cells upon administration of a given dose of a substance to a mammal among a set of mammals of which is each given a different dose of the material, could be used to determine a starting dose to be administered to a mammal. The extent to which the tumor cells are killed upon administration of a certain dose can be assayed by methods known in the art.

The dose also can be determined by the existence, nature and extent of any adverse side effects that might accompany the administration. A variety of factors, such as age, body weight, general health, diet, sex, material to be administered, route of administration, dosing schedule and the severity of the condition being treated can be considered when determining dosage.

The composition can be administered intravenously in a wide dosing range from about 0.01 milligram per kilo body weight (mg/kg) to about 1.0 mg/kg, depending on patient's age and physical state, as well as dosing regimen and schedule.

In some embodiments the composition is lyophilized in 20 mM glutamate, 10 mg/mL trehalose, 30 mg/mL mannitol, pH 4.5 and reconstituted in sterile water prior to use. In another embodiment the composition is lyophilized in 20 mM histidine, 10 mg/mL trehalose, 30 mg/mL mannitol, pH 6.5 and reconstituted in sterile water prior to use. In yet another embodiment, the composition is dissolved in 20 mM histidine, 150 mM NaCl pH 6.5 and kept frozen prior to use.

### II. Compositions

Compositions for inducing, increasing, enhancing or promoting an immune response are provided. In some embodiments, the disclosed compositions cause an increase in mitochondrial number, can increase in mitochondrial respiration relative to a control, or both. The composition typically includes an effective amount of a mitochondrial DNA-binding polypeptide. Examples of a mitochondrial DNA-binding polypeptides include, but are not limited to, mitochondrial transcription factors such as transcription factor A, mitochondrial (TFAM) having GenBank Accession No. mitochondrial NM_{—}003201; transcription factor B1, mitochondrial (TFB1M) having GenBank Accession No. AF151833; transcription factor B2, mitochondrial (TFB2M) having GenBank Accession No. AK026835; Polymerase (RNA) Mitochondrial (DNA directed) (POLRMT) having GenBank Accession No. NM_{—}005035; and functional fragments, variants, and fusion polypeptides thereof.

In preferred embodiments the composition includes a recombinant fusion protein including a polynucleotide-binding polypeptide, a protein transduction domain, and optionally one or more targeting signals. In some embodiments, the disclosed compositions cause an increase in mitochondrial number, an increase in mitochondrial respiration, an increase mitochondrial Electron Transport Chain (ETC) activity, increased oxidative phosphorylation, increased oxygen consumption, increased ATP production, or combinations thereof relative to a control. In preferred embodiments the composition reduces oxidative stress.

Exemplary fusion proteins containing a mitochondrial transcription factor polypeptide are disclosed in U.S. Pat. Nos. 8,039,587, 8,062,891, 8,133,733, and U.S. Published Application Nos. 2009/0123468, 2009/0208478, and 2006/0211647 all of which are specifically incorporated by reference herein in their entireties.

A. Polypeptides

1. Polynucleotide Binding Domain

The compositions for inducing, increasing, enhancing or promoting an immune response include an effective amount of a mitochondrial DNA-binding polypeptide optionally having a PTD and optionally having one or more targeting signals or domains. In certain embodiments, the mitochondrial DNA-binding polypeptide is a polypeptide known to bind or package a mtDNA. Preferably, the mitochondrial DNA-binding polypeptide is a recombinant polypeptide. The recombinant polypeptide can be used as a therapeutic agent either alone or in combination with a polynucleotide, or any other active agent. In preferred embodiments the polynucleotide-binding domain includes mature TFAM, a functional fragment of TFAM, or a variant thereof. In certain embodiments, the polynucleotide-binding polypeptide includes at least a portion of a member of the high mobility group (HMG) of proteins effective to bind a polynucleotide, for example an HMG box domain.

“Mature TFAM” refers to TFAM after it has been post-translationally modified and is in the form that is active in the mitochondrion. For example, a mature TFAM is one in which the endogenous mitochondrial signal sequence has been cleaved.

a. Transcription Factor A, Mitochondria (TFAM)

One embodiment provides a non-histone polynucleotide-binding polypeptide, for example mitochondrial transcription factor A (TFAM) polypeptide, for functional fragment, or a variant thereof. Variant TFAM can have 80%, 85%, 90%, 95%, 99% or greater sequence identity with a reference TFAM, for example naturally occurring TFAM having GenBank Accession No. NM_{—}003201. In certain embodiments, the variant TFAM has 80%, 85%, 90%, 95%, 99% or greater sequence identity with a reference TFAM. In certain embodiments, the variant TFAM has 80%, 85%, 90%, 95%, 99% or greater sequence identity over the full-length of mature human TFAM.

TFAM is a member of the high mobility group (HMG) of proteins having two HMG-box domains. TFAM as well as other HMG proteins bind, wrap, bend, and unwind DNA. Thus, embodiments of the present disclosure include polynucleotide binding polypeptides including one or more polynucleotide binding regions of the HMG family of proteins, and optionally induce a structural change in the polynucleotide when the polypeptide binds or becomes associated with the polynucleotide.

In some embodiment, the polynucleotide-binding polypeptide is full-length TFAM polypeptide, or variant therefore. For example, a preferred TFAM polypeptide has at least 80, 85, 90, 95, 99, or 100 percent sequence identity to the full-length TFAM precursor

(SEQ ID NO: 1)
MAFLRSMWGV LSALGRSGAE LCTGCGSRLR SPFSFVYLPR
WFSSVLASCP KKPVSSYLRF SKEQLPIFKA QNPDAKTTEL
IRRIAQRWRE LPDSKKKIYQ DAYRAEWQVY KEEISRFKEQ
LTPSQIMSLE KEIMDKHLKR KAMTKKKELT LLGKPKRPRS
AYNVYVAERF QEAKGDSPQE KLKTVKENWK NLSDSEKELY
IQHAKEDETR YHNEMKSWEE QMIEVGRKDL LRRTIKKQRK
YGAEEC.

Many nuclear encoded mitochondrial proteins destined for the mitochondrial matrix are translated as a “preprotein.” The preprotein sequence includes a signal peptide as known as an “amino-terminal signal”, or a “presequence” that facilitates translocation from the cytosol through the mitochondrial translocation machinery in the outer membrane called the Tom complex (Translocator outer membrane) as well as the machinery in the inner membrane called the Tim complex (Translocator Inner Membrane). Once the preprotein enters the inner mitochondrial matrix, the signal sequence is cleaved by a protease such as MPP. A mitochondrial protein with the signal sequence cleaved or removed can be referred to as a “mature” protein. Therefore, in some embodiments, the polynucleotide-binding polypeptide is a mature TFAM polypeptide, or variant thereof. For example, in some embodiments, the cleavable mitochondrial targeting sequence of a TFAM preprotein is amino acid residue 1 of SEQ ID NO:1 to amino acid residue 42 of SEQ ID NO:1,

(SEQ ID NO: 2)
MAFLRSMWGV LSALGRSGAE LCTGCGSRLR
SPFSFVYLPR WF.

In certain embodiments, a preferred TFAM polypeptide has at least 80, 85, 90, 95, 99, or 100 percent sequence identity to the mature TFAM sequence

(SEQ ID NO: 3)
SSVLASCPKK PVSSYLRFSK EQLPIFKAQN PDAKTTELIR
RIAQRWRELP DSKKKIYQDA YRAEWQVYKE EISRFKEQLT
PSQIMSLEKE IMDKHLKRKA MTKKKELTLL GKPKRPRSAY
NVYVAERFQE AKGDSPQEKL KTVKENWKNL SDSEKELYIQ
HAKEDETRYH NEMKSWEEQM IEVGRKDLLR RTIKKQRKYG AEEC.

In some embodiments, the polynucleotide-binding polypeptide is functional fragment of TFAM, or variant therefore. A functional fragment of TFAM as used herein is a fragment of full-length TFAM that is when administered to a patient reduces, inhibits or alleviates, one or more symptoms or sides effects associated with physical insult caused by or chemotherapy or high levels of radiation compared to a control. Functional fragments can be effective when administered alone, or can be effective when administered in combination with a polynucleotide. Functional fragments of TFAM can include, but are not limited to, a fragment of full-length TFAM sufficient to bind non-specifically to a polynucleotide, a fragment of full-length TFAM sufficient to bind specifically to the mtDNA light strand promoter (LSP), the mtDNA heavy strand promoter 1 (HSP1), the mtDNA heavy stand promoter 2 (HSP2), or combinations thereof, a fragment of full-length TFAM sufficient to induce mitochondrial transcription, a fragment of full-length TFAM sufficient to induce oxidative phosphorylation, a fragment of full-length TFAM sufficient to induce mitochondrial biogenesis, and combinations thereof.

From N-terminus to C-terminus, mature TFAM includes four domains, a first HMG box (also referred to herein as HMG box 1), followed by a linker region (also referred to herein as linker), followed by a second HMG box (also referred to herein as HMG box 2), followed by a C-terminal tail. Functional fragments of TFAM typically include one or more domains of mature TFAM, or a variant thereof. For example, in some embodiments, the functional fragment includes one or more HMG box 1 domains of TFAM, one or more linker domains of TFAM, one or more HMG box 2 domains of TFAM, one or more C-terminal tail domains of TFAM, or combinations thereof. The domains can be arranged in the same orientation of the domains of endogenous TFAM, or they can be rearranged so they are in a different order or orientation than the domains found in endogenous TFAM protein. In certain embodiments the functional fragment includes a first HMG box domain, and second HMG box domain linked to the first HMG box domain with a linker, typically a peptide linker. The linker can be the endogenous linker domain of TFAM, or a heterologous linker that allows the first and the second HMG box domains to maintain their functional activity. Deletion studies characterizing the activity of different domains and hybrid constructs of TFAM are known in the art and characterized for example in Dairaghi, et al., J. Mol. Biol., 249:11-28 (1995), Matsushima, et al., J. Biol. Chem., 278(33):31149-31158 (2003), and Gangeloff, et al., Nucl. Acid. Res., 37(10):3153-3164 (2009), all of which are specifically incorporated by reference herein in their entireties.

In certain embodiments a functional fragment is one or more domains of TFAM according to SEQ ID NO: 3. For example, an HMG box 1 of TFAM can be a polypeptide including the sequence from amino acid residue 1, 2, 3, 4, 5, 6, 7, 8, 9, or 10 of SEQ ID NO: 3 to amino acid residue 70, 71, 72, 73, 74, 75, 76, 77, 78, 79, 80, 81, 82, 83, 84, 85, 86, 87, 88, 89, 90, 91, 92, 93, 94, or 95 of SEQ ID NO: 3, or a variant thereof with 80, 85, 90, 95, 99, or greater than 99 percent sequence identity to the corresponding fragment of SEQ ID NO: 3.

A linker region of TFAM can be a polypeptide including the sequence from amino acid residue 70, 71, 72, 73, 74, 75, 76, 77, 78, 79, 80, 81, 82, 83, 84, or 85 of SEQ ID NO: 3 to amino acid residue 105, 106, 107, 108, 109, 110, 111, 112, 113, 114, or 115 of SEQ ID NO: 3, or a variant thereof with 80, 85, 90, 95, 99, or greater than 99 percent sequence identity to the corresponding fragment of SEQ ID NO: 3.

An HMG box 2 of TFAM can be a polypeptide including the sequence from amino acid residue 96, 97, 98, 99, 100, 101, 102, 103, 104, 105, 106, 107, 108, 109, 110, 111, 112, 113, 114, or 115 of SEQ ID NO: 3 to amino acid residue 172, 173, 174, 175, 176, 177, 178, 179, 180, 181, 182, 183, 184, 185, 186, or 187 of SEQ ID NO: 3, or a variant thereof with 80, 85, 90, 95, 99, or greater than 99 percent sequence identity to the corresponding fragment of SEQ ID NO: 3.

A C-terminal tail of TFAM can be a polypeptide including the sequence from amino acid residue 172, 173, 174, 175, 176, 177, 178, 179, 180, 181, 182, 183, 184, 185, 186, or 187 of SEQ ID NO: 3 to amino acid residue 194, 195, 196, 197, 198, 199, 200, 201, 202, 203, or 204 of SEQ ID NO: 3, or a variant thereof with 80, 85, 90, 95, 99, or greater than 99 percent sequence identity to the corresponding fragment of SEQ ID NO: 3.

Variants of TFAM and functional fragments of TFAM are also provided. Typically, the variants of TFAM and function fragments of TFAM include one or more conservative amino acid substitutions relative to the corresponding reference sequence, for example SEQ ID NO:3, or a fragment thereof. One embodiment provides a TFAM polypeptide having one or more serine residues at positions 1, 2 and 13 SEQ ID NO:3 substituted with an alanine or threonine residue. A preferred embodiment provides a TFAM polypeptide having serine 13 of SEQ ID NO:3 substituted for an alanine or threonine. The variant TFAM polypeptides have improved mtDNA binding in the presence of glucose or elevated glucose levels.

Selected model organisms that have TFAM sequences that are useful in the compositions and methods disclosed herein include, but are not limited to those disclosed in Table 1:

TABLE 1
Organism, Protein And Percent Identity And Length Of Aligned Region

| | | |
|---|---|---|
| H. sapiens | sp: Q00059 - MTT1_HUMAN | 100%/246 aa |
| | Transcription factor 1, mitochondrial | (see ProtEST) |
| | precursor (MTTF1) | |
| M. musculus | ref: NP_033386.1 - transcription | 63%/237 aa |
| | factor A, mitochondrial | (see ProtEST) |
| | [Mus musculus] | |
| R. norvegicus: | ref: NP_112616.1 - transcription | 64%/237 aa |
| | factor A, mitochondrial | |
| | [Rattus norvegicus] | (see ProtEST) |
| A. thaliana | ref: NP_192846.1 - 98b like | 27%/189 aa |
| | protein [Arabidopsis thaliana] | (see ProtEST) |
| C. elegans | ref: NP_501245.1 - F45E4.9.p | 27%/189 aa |
| | [Caenorhabditis elegans] | (see ProtEST) |
| D. melanogaster: | ref: NP_524415.1 - | 34%/183 aa |
| | mitochondrial transcription | (see ProtEST) |
| | factor A [Drosophila melanogaster] | |

b. Transcription Factor B1, Mitochondrial (TFB1M)

The polynucleotide-binding polypeptide can be transcription factor B1, mitochondrial (TFB1M). A preferred TFB1M has GenBank Accession No. AF151833. TFB1 is part of the complex involved in mitochondrial transcription. The process of transcription initiation in mitochondria involves three types of proteins: the mitochondrial RNA polymerase (POLRMT), mitochondrial transcription factor A (TFAM), and mitochondrial transcription factors B1 and B2 (TFB1M, TFB2M). POLRMT, TFAM, and TFB1M or TFB2M assemble at the mitochondrial promoters and begin transcription. TFB1M has about 1/10 the transcriptional activity of TFB2M, and both TFBs are also related to rRNA methyltransferases and TFB1M can bind S-adenosylmethionine and methylate mitochondrial 12S rRNA. Additionally, TFB1M and TFB2M can bind single-stranded nucleic acids.

A preferred TFB1M polypeptide has at least 80, 85, 90, 95, 99, or 100 percent sequence identity to

(SEQ ID NO: 4)
MAASGKLSTC RLPPLPTIRE IIKLLRLQAA NELSQNFLLD
LRLTDKIVRK AGNLTNAYVY EVGPGPGGIT RSILNADVAE
LLVVEKDTRF IPGLQMLSDA APGKLRIVHG DVLTFKVEKA
FSESLKRPWE DDPPNVHIIG NLPFSVSTPL IIKWLENISC
RDGPFVYGRT QMTLTFQKEV AERLAANTGS KQRSRLSVMA
QYLCNVRHIF TIPGQAFVPK PEVDVGVVHF TPLIQPKIEQ
PFKLVEKVVQ NVFQFRRKYC HRGLRMLFPE AQRLESTGRL
LELADIDPTL RPRQLSISHF KSLCDVYRKM CDEDPQLFAY
NFREELKRRK SKNEEKEEDD AENYRL.

c. Transcription Factor B2, Mitochondrial (TFB2M)

In still another embodiment, the polynucleotide-binding polypeptide includes TFB2M. In a preferred embodiment the TFB2M polypeptide has GenBank Accession No. AK026835. TFB2M also possesses a Rossmann-fold making it part of the NAD-binding protein family. TFB2M levels modulate mtDNA copy number and levels of mitochondrial transcripts as would be expected of a mitochondrial transcription factor. It is appreciated by those skilled in the art that increased activity of mitochondria causes an increase in mitochondrial biogenesis.

A preferred TFB2M polypeptide has at least 80, 85, 90, 95, 99, or 100 percent sequence identity to

(SEQ ID NO: 5)
MWIPVVGLPR RLRLSALAGA GRFCILGSEA ATRKHLPARN
HCGLSDSSPQ LWPEPDFRNP PRKASKASLD FKRYVTDRRL
AETLAQIYLG KPSRPPHLLL ECNPGPGILT QALLEAGAKV
VALESDKTFI PHLESLGKNL DGKLRVIHCD FFKLDPRSGG
VIKPPAMSSR GLFKNLGIEA VPWTADIPLK VVGMFPSRGE
KRALWKLAYD LYSCTSIYKF GRIEVNMFIG EKEFQKLMAD
PGNPDLYHVL SVIWQLACEI KVLHMEPWSS FDIYTRKGPL
ENPKRRELLD QLQQKLYLIQ MIPRQNLFTK NLTPMNYNIF
FHLLKHCFGR RSATVIDHLR SLTPLDARDI LMQIGKQEDE
KVVNMHPQDF KTLFETIERS KDCAYKWLYD ETLEDR.

d. Polymerase (RNA) Mitochondrial (DNA Directed) (POLRMT)

Still another polynucleotide-binding polypeptide that can be used to modulate mitochondrial biological activity is POLRMT. In a preferred embodiment, the POLRMT polypeptide has GenBank Accession No. NM_{—}005035. POLRMT is a mitochondrial RNA polymerase similar in structure to phage RNA polymerases. Unlike phage polymerases, POLRMT contains two pentatricopeptide repeat (PPR) domains involved in regulating mitochondrial transcripts. It is appreciated by those skilled in the art that deletion of regulatory domains enables constitutive function.

A preferred POLRMT polypeptide has at least 80, 85, 90, 95, 99, or 100 percent sequence identity to

(SEQ ID NO: 39)
MSALCWGRGA AGLKRALRPC GRPGLPGKEG TAGGVCGPRR
SSSASPQEQD QDRRKDWGHV ELLEVLQARV RQLQAESVSE
VVVNRVDVAR LPECGSGDGS LQPPRKVQMG AKDATPVPCG
RWAKILEKDK RTQQMRMQRL KAKLQMPFQS GEFKALTRRL
QVEPRLLSKQ MAGCLEDCTR QAPESPWEEQ LARLLQEAPG
KLSLDVEQAP SGQHSQAQLS GQQQRLLAFF KCCLLTDQLP
LAHHLLVVHH GQRQKRKLLT LDMYNAVMLG WARQGAFKEL
VYVLFMVKDA GLTPDLLSYA AALQCMGRQD QDAGTIERCL
EQMSQEGLKL QALFTAVLLS EEDRATVLKA VHKVKPTFSL
PPQLPPPVNT SKLLRDVYAK DGRVSYPKLH LPLKTLQCLF
EKQLHMELAS RVCVVSVEKP TLPSKEVKHA RKTLKTLRDQ
WEKALCRALR ETKNRLEREV YEGRFSLYPF LCLLDEREVV
RMLLQVLQAL PAQGESFTTL ARELSARTFS RHVVQRQRVS
GQVQALQNHY RKYLCLLASD AEVPEPCLPR QYWEELGAPE
ALREQPWPLP VQMELGKLLA EMLVQATQMP CSLDKPHRSS
RLVPVLYHVY SFRNVQQIGI LKPHPAYVQL LEKAAEPTLT
FEAVDVPMLC PPLPWTSPHS GAFLLSPTKL MRTVEGATQH
QELLETCPPT ALHGALDALT QLGNCAWRVN GRVLDLVLQL
FQAKGCPQLG VPAPPSEAPQ PPEAHLPHSA APARKAELRR
ELAHCQKVAR EMHSLRAEAL YRLSLAQHLR DRVFWLPHNM
DFRGRTYPCP PHFNHLGSDV ARALLEFAQG RPLGPHGLDW
LKIHLVNLTG LKKREPLRKR LAFAEEVMDD ILDSADQPLT
GRKWWMGAEE PWQTLACCME VANAVRASDP AAYVSHLPVH
QDGSCNGLQH YAALGRDSVG AASVNLEPSD VPQDVYSGVA
AQVEVFRRQD AQRGMRVAQV LEGFITRKVV KQTVMTVVYG
VTRYGGRLQI EKRLRELSDF PQEFVWEASH YLVRQVFKSL
QEMFSGTRAI QHWLTESARL ISHMGSVVEW VTPLGVPVIQ
PYRLDSKVKQ IGGGIQSITY THNGDISRKP NTRKQKNGFP
PNFIHSLDSS HMMLTALHCY RKGLTFVSVH DCYWTHAADV
SVMNQVCREQ FVRLHSEPIL QDLSRFLVKR FCSEPQKILE
ASQLKETLQA VPKPGAFDLE QVKRSTYFFS.

e. HMG Domain

In some embodiments, the polynucleotide-binding polypeptide is a non-TFAM HMG domain. Generally, the HMG domain includes a global fold of three helices stabilized in an ‘L-shaped’ configuration by two hydrophobic cores. The high mobility group chromosomal proteins HMG1 or HMG2, which are common to all eukaryotes, bind DNA in a non-sequence-specific fashion, for example to promote chromatin function and gene regulation. They can interact directly with nucleosomes and are believed to be modulators of chromatin structure. They are also important in activating a number of regulators of gene expression, including p53, Hox transcription factors and steroid hormone receptors, by increasing their affinity for DNA. HMG proteins include HMG-1/2, HMG-I(Y) and HMG-14/17.

The HMG-1/2-box proteins can be further distinguished into three subfamilies according to the number of HMG domains present in the protein, their specific of sequence recognition and their evolutionary relationship. The first group contains chromosomal proteins bound to DNA with no sequence specificity (class I, HMG1 and HMG2), the second contains ribosomal and mitochondrial transcription factors which show sequence specificity in the presence of another associating factor when bound with DNA (class II, yeast ARS binding protein ABF-2, UBF and mitochondrial transcription factor mtTF-1), and the third contains gene-specific transcription factors which show sequence specific DNA binding (class III, lymphoid enhancer-binding factors LEF-1 and TCF-1; the mammalian sex-determining factor SRY, and the closely related SOX proteins; and the fungal regulatory proteins Mat-MC, Mat-a1, Stel1 and Rox1). The HMG1/2-box DNA binding domain is about 75 to about 80 amino acids and contains highly conserved proline, aromatic and basic residues. Common properties of HMG domain proteins include interaction with the minor groove of the DNA helix, binding to irregular DNA structure, and the capacity to modulate DNA structure by bending.

SOX (SRY-type HMG box) proteins have critical functions in a number of developmental processes, including sex determination, skeleton formation, pre-B and T cell development and neural induction. SOX9 plays a direct role during chondrogenesis by binding and activating the chondrocyte-spacific enhancer of the Col2a1 gene. Loss of SOX9 gene function leads to the genetic condition known as Campomelic Dysplsia (CD), a form of dwarfism characterized by extreme skeletal malformation, and one in which three-quarters of XY individual are either intersexes or exhibit male to female sex reversal. There are more than 20 members cloned in SOX family. All of which contain an HMG domain, which can bind specifically to the double strand DNA motif and shares >50% identify with the HMG domain of SRY, the human testis-determining factor. The preferred DNA-binding site of SOX9 have been defined to be

(SEQ ID NO: 6)
AGAACAATGG,

which contains the SOX core-binding element (SCBE), AACAAT, flanking 5′ AG and 3′ GG nucleotides enhance binding by SOX9.

In one embodiment, the recombinant polynucleotide-binding polypeptide has at least one HMG box domain, generally at least two, more particularly 2-5 HMG box domains. The HMG box domain can bind to an AT rich DNA sequence, for example, using a large surface on the concave face of the protein, to bind the minor groove of the DNA. This binding bends the DNA helix axis away from the site of contact. The first and second helices contact the DNA, their N-termini fitting into the minor groove whereas helix 3 is primarily exposed to solvent. Partial intercalation of aliphatic and aromatic residues in helix 2 occurs in the minor groove.

In other embodiments, the polynucleotide-binding polypeptide can have at least one polynucleotide binding domain, typically two or more polynucleotide binding domains. The polynucleotide binding domains can be the same or different. For example, the polynucleotide-binding polypeptide can include at least one HMG box in combination with one or more DNA binding domains selected from the group consisting of an HMG box, homeodomain and POU domain; zinc finger domain such as C₂H₂ and C₂C₂; amphipathic helix domain such as leucine zipper and helix-loop-helix domains; and histone folds. The polynucleotide binding domain can be specific for a specific polynucleotide sequence, or preferably non-specifically binds to a polynucleotide. Alternatively, the polynucleotide-binding polypeptide can have more a combination of at least one polynucleotide binding domain that binds in a sequence specific manner and at least one polynucleotide binding-domain that binds DNA non-specifically.

f. Helix-Turn-Helix

Certain embodiments provide polynucleotide-binding polypeptides having a helix-turn-helix motif or at least a polynucleotide binding region of a helix-turn-helix protein. Helix-turn-helix proteins have a similar structure to bacterial regulatory proteins such as the 1 repressor and cro proteins, the lac repressor and so on which bind as dimers and their binding sites are palindromic. They contain 3 helical regions separated by short turns which is why they are called helix-turn-helix proteins. One protein helix (helix 3) in each subunit of the dimer occupies the major groove of two successive turns of the DNA helix. Thus, in another embodiment, the disclosed polynucleotide-binding polypeptides can form dimers or other multi-component complexes, and have 1 to 3 helices.

g. Homeodomain

In yet another embodiment, the polynucleotide-binding polypeptide includes a homeodomain or a portion of a homeodomain protein. Homeodomain proteins bind to a sequence of 180 base pairs initially identified in a group of genes called homeotic genes. Accordingly, the sequence was called the homeobox. The 180 bp corresponds to 60 amino acids in the corresponding protein. This protein domain is called the homeodomain. Homeodomain-containing proteins have since been identified in a wide range of organisms including vertebrates and plants. The homeodomain shows a high degree of sequence conservation. The homeodomain contains 4 α helical regions. Helices II and III are connected by 3 amino acids comprising a turn. This region has a very similar structure to helices II and III of bacterial DNA binding proteins.

h. Zinc Finger

Yet another embodiment provides a modified polynucleotide-binding polypeptide having a zinc finger domain or at least a portion of a zinc finger protein. Zinc finger proteins have a domain with the general structure: Phe (sometimes Tyr)—Cys—2 to 4 amino acids—Cys—3 amino acids—Phe (sometimes Tyr)—5 amino acids—Leu—2 amino acids—His—3 amino acids—His. The phenylalanine or tyrosine residues which occur at invariant positions are required for DNA binding. Similar sequences have been found in a range of other DNA binding proteins though the number of fingers varies. For example, the SP1 transcription factor which binds to the GC box found in the promoter proximal region of a number of genes has 3 fingers. This type of zinc finger which has 2 cysteines and 2 histidines is called a C₂H₂ zinc finger.

Another type of zinc finger which binds zinc between 2 pairs of cysteines has been found in a range of DNA binding proteins. The general structure of this type of zinc finger is: Cys—2 amino acids—Cys—13 amino acids—Cys—2 amino acids—Cys. This is called a C₂C₂ zinc finger. It is found in a group of proteins known as the steroid receptor superfamily, each of which has 2 C₂C₂ zinc fingers.

i. Leucine Zipper

Another embodiment provides a modified polynucleotide-binding polypeptide having a leucine zipper or at least a portion of a leucine zipper protein. The first leucine zipper protein was identified from extracts of liver cells, and it was called C/EBP because it is an enhancer binding protein and it was originally thought to bind to the CAAT promoter proximal sequence. C/EBP will only bind to DNA as a dimer. The region of the protein where the two monomers join to make the dimer is called the dimerization domain. This lies towards the C-terminal end of the protein. When the amino acid sequence was examined it was found that a leucine residue occurs every seventh amino acid over a stretch of 35 amino acids. If this region were to form a helix then all of these leucines would align on one face of the helix.

Because leucine has a hydrophobic side chain, one face of the helix is very hydrophobic. The opposite face has amino acids with charged side chains which are hydrophilic. The combination of hydrophobic and hydrophilic characteristics gives the molecule is amphipathic moniker. Adjacent to the leucine zipper region is a region of 20-30 amino acids which is rich in the basic (positively charged) amino acids lysine and arginine. This is the DNA binding domain—often referred to as the bZIP domain—the basic region of the leucine zipper. C/EBP is thought to bind to DNA by these bZIP regions wrapping round the DNA helix

The leucine zipper—bZIP structure has been found in a range of other proteins including the products of the jun and fos oncogenes. Whereas C/EBP binds to DNA as a homodimer of identical subunits, fos cannot form homodimers at all and jun/jun homodimers tend to be unstable. However fos/jun heterodimers are much more stable. These fos/jun heterodimers correspond to a general transcription factor called AP1 which binds to a variety of promoters and enhancers and activates transcription. The consensus AP1 binding site is TGACTCA which is palindromic.

j. Helix-Loop-Helix

Another embodiment provides a modified polynucleotide-binding polypeptide having helix-loop-helix domain or a polynucleotide binding portion of a helix-loop-helix protein. Helix-loop-helix proteins are similar to leucine zippers in that they form dimers via amphipathic helices. They were first discovered as a class of proteins when a region of similarity was noticed between two enhancer binding proteins called E47 and E12. This conserved region has the potential to form two amphipathic separated by a loop hence helix-loop-helix. Next to the dimerization domain is a DNA binding domain, again rich in basic amino acids and referred to as the bHLH domain. These structures are also found in a number of genes required for development of the Drosophila nervous system—the Achaete-scute complex, and in a protein called MyoD which is required for mammalian muscle differentiation.

k. Histone Fold

In still another embodiment, the modified polynucleotide-binding polypeptide includes a histone polypeptide, a fragment of a histone polypeptide, or at least one histone fold. Histone folds exist in histone polypeptides monomers assembled into dimers. Histone polypeptides include H2A, H2B, H3, and H4 which can form heterodimers H2A-2B and H3-H4. It will be appreciated that histone-like polypeptides can also be used in the disclosed compositions and methods. Histone-like polypeptides include, but are not limited to, HMf or the histone from Methanothermous fervidus, other archaeal histones known in the art, and histone-fold containing polypeptides such as MJ1647, CBF, TAFII or transcription factor IID, SPT3, and Dr1-DRAP (Sanderman, K., et al., Cell. Mol. Life Sci. 54:1350-1364 (1998), which is specifically incorporated by reference herein in its entirety).

2. Protein Transduction Domain

In some embodiments, the polynucleotide-binding polypeptide is fusion protein modified to include a protein transduction domain (PTD). As used herein, a “protein transduction domain” or PTD refers to a polypeptide, polynucleotide, carbohydrate, organic or inorganic compound that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle.

In preferred embodiments, the protein transduction domain is a polypeptide. A protein transduction domain can be a polypeptide including positively charged amino acids. Thus, some embodiments include PTDs that are cationic or amphipathic. Protein transduction domains (PTD), also known as a cell penetrating peptides (CPP), are typically polypeptides including positively charged amino acids. PTDs are known in the art, and include but are not limited to small regions of proteins that are able to cross a cell membrane in a receptor-independent mechanism (Kabouridis, P., Trends in Biotechnology (11):498-503 (2003)). Although several PTDs have been documented, the two most commonly employed PTDs are derived from TAT (Frankel and Pabo, Cell, 55(6):1189-93 (1988)) protein of HIV and Antennapedia transcription factor from Drosophila, whose PTD is known as Penetratin (Derossi et al., J Biol Chem., 269(14):10444-50 (1994)). Exemplary protein transduction domains include polypeptides with 11 Arginine residues, or positively charged polypeptides or polynucleotides having 8-15 residues, preferably 9-11 residues.

The Antennapedia homeodomain is 68 amino acid residues long and contains four alpha helices. Penetratin is an active domain of this protein which consists of a 16 amino acid sequence derived from the third helix of Antennapedia. TAT protein consists of 86 amino acids and is involved in the replication of HIV-1. The TAT PTD consists of an 11 amino acid sequence domain (residues 47 to 57;

(SEQ ID NO: 7)
YGRKKRRQRR R)

of the parent protein that appears to be critical for uptake. Additionally, the basic domain Tat(49-57) or

(SEQ ID NO: 8)
RKKRRQRRR

has been shown to be a PTD. In the current literature TAT has been favored for fusion to proteins of interest for cellular import. Several modifications to TAT, including substitutions of Glutatmine to Alanine, i.e., A, have demonstrated an increase in cellular uptake anywhere from 90% (Wender et al., Proc Natl Acad Sci USA., 97(24):13003-8 (2000)) to up to 33 fold in mammalian cells. (Ho et al., Cancer Res., 61(2):474-7 (2001)).

The most efficient uptake of modified proteins was revealed by mutagenesis experiments of TAT-PTD, showing that an 11 arginine stretch was several orders of magnitude more efficient as an intercellular delivery vehicle. Therefore, PTDs can include a sequence of multiple arginine residues, referred to herein as poly-arginine or poly-ARG. In some embodiments the sequence of arginine residues is consecutive. In some embodiments the sequence of arginine residues is non-consecutive. A poly-ARG can include at least 7 arginine residues, more preferably at least 8 arginine residues, most preferably at least 11 arginine residues. In some embodiments, the poly-ARG includes between 7 and 15 arginine residues, more preferably between 8 and 15 arginine residues. In some embodiments the poly-ARG includes between 7 and 15, more preferably between 8 and 15 consecutive arginine residues. An example of a poly-ARG is

(SEQ ID NO: 9)
RRRRRRR.

Additional exemplary PTDs include but are not limited to;

(SEQ ID NO: 10)
RRQRRTSKLM KR;
(SEQ ID NO: 11)
GWTLNSAGYL LGKINLKALA ALAKKIL;
(SEQ ID NO: 12)
WEAKLAKALA KALAKHLAKA LAKALKCEA;
and
(SEQ ID NO: 13)
RQIKIWFQNR RMKWKK.

Without being bound by theory, it is believed that following an initial ionic cell-surface interaction, some polypeptides containing a protein transduction domain are rapidly internalized by cells via lipid raft-dependent macropinocytosis. For example, transduction of a TAT-fusion protein was found to be independent of interleukin-2 receptor/raft-, caveolar- and clathrin-mediated endocytosis and phagocytosis (Wadia, et al., Nature Medicine, 10:310-315 (2004), and Barka, et al., J. Histochem. Cytochem., 48(11):1453-60 (2000)). Therefore, in some embodiments the polynucleotide-binding polypeptide includes an endosomal escape sequence that enhances escape of the polypeptide-binding protein from macropinosomes. The some embodiments the endosomal escape sequence is part of, or consecutive with, the protein transduction domain. In some embodiments, the endosomal escape sequence is non-consecutive with the protein transduction domain. In some embodiments the endosomal escape sequence includes a portion of the hemagglutinin peptide from influenza (HA). One example of an endosomal escape sequence includes

(SEQ ID NO: 14)
GDIMGEWG NEIFGAIAGF LG.

In one embodiment a protein transduction domain including an endosomal escape sequence includes the amino acid sequence

(SEQ ID NO: 15)
RRRRRRRRRR RGEGDIMGEW GNEIFGAIAG FLGGE.

3. Targeting Signal or Domain

In some embodiments the polynucleotide-binding polypeptide is modified to include one or more targeting signals or domains. The targeting signal can include a sequence of monomers that facilitates in vivo localization of the molecule. The monomers can be amino acids, nucleotide or nucleoside bases, or sugar groups such as glucose, galactose, and the like which form carbohydrate targeting signals. Targeting signals or sequences can be specific for a host, tissue, organ, cell, organelle, non-nuclear organelle, or cellular compartment. For example, in some embodiments the polynucleotide-binding polypeptide includes both a cell-specific targeting domain and an organelle specific targeting domain to enhance delivery of the polypeptide to a subcellular organelle of a specific cells type.

i. Organelle Targeting

In some embodiments, the polynucleotide-binding polypeptide is modified to target a subcellular organelle. Targeting of the disclosed polypeptides to organelles can be accomplished by modifying the disclosed compositions to contain specific organelle targeting signals. These sequences can target organelles, either specifically or non-specifically. In some embodiments the interaction of the targeting signal with the organelle does not occur through a traditional receptor:ligand interaction.

The eukaryotic cell comprises a number of discrete membrane bound compartments, or organelles. The structure and function of each organelle is largely determined by its unique complement of constituent polypeptides. However, the vast majority of these polypeptides begin their synthesis in the cytoplasm. Thus organelle biogenesis and upkeep require that newly synthesized proteins can be accurately targeted to their appropriate compartment. This is often accomplished by amino-terminal signaling sequences, as well as post-translational modifications and secondary structure.

Organelles can have single or multiple membranes and exist in both plant and animal cells. Depending on the function of the organelle, the organelle can consist of specific components such as proteins and cofactors. The polypeptides delivered to the organelle can enhance or contribute to the functioning of the organelle. Some organelles, such as mitochondria and chloroplasts, contain their own genome. Nucleic acids are replicated, transcribed, and translated within these organelles. Proteins are imported and metabolites are exported. Thus, there is an exchange of material across the membranes of organelles. Exemplary organelles include the nucleus, mitochondrion, chloroplast, lysosome, peroxisome, Golgi, endoplasmic reticulum, and nucleolus. Synthetic organelles can be formed from lipids and can contain specific proteins within the lipid membranes. Additionally, the content of synthetic organelles can be manipulated to contain components for the translation of nucleic acids.

a. Targeting the Mitochondria

In certain embodiments polynucleotide-binding polypeptides are disclosed that specifically target mitochondria. Mitochondria contain the molecular machinery for the conversion of energy from the breakdown of glucose into adenosine triphosphate (ATP). The energy stored in the high energy phosphate bonds of ATP is then available to power cellular functions. Mitochondria are mostly protein, but some lipid, DNA and RNA are present. These generally spherical organelles have an outer membrane surrounding an inner membrane that folds (cristae) into a scaffolding for oxidative phosphorylation and electron transport enzymes. Most mitochondria have flat shelf-like cristae, but those in steroid secreting cells may have tubular cristae. The mitochondrial matrix contains the enzymes of the citric acid cycle, fatty acid oxidation and mitochondrial nucleic acids.

Mitochondrial DNA is double stranded and circular. Mitochondrial RNA comes in the three standard varieties; ribosomal, messenger and transfer, but each is specific to the mitochondria. Some protein synthesis occurs in the mitochondria on mitochondrial ribosomes that are different than cytoplasmic ribosomes. Other mitochondrial proteins are made on cytoplasmic ribosomes with a signal peptide that directs them to the mitochondria. The metabolic activity of the cell is related to the number of cristae and the number of mitochondria within a cell. Cells with high metabolic activity, such as heart muscle, have many well developed mitochondria. New mitochondria are formed from preexisting mitochondria when they grow and divide.

The inner membranes of mitochondria contain a family of proteins of related sequence and structure that transport various metabolites across the membrane. Their amino acid sequences have a tripartite structure, made up of three related sequences about 100 amino acids in length. The repeats of one carrier are related to those present in the others and several characteristic sequence features are conserved throughout the family.

Mitochondrial targeting agents generally consist of a leader sequence of highly positively charged amino acids. This allows the protein to be targeted to the highly negatively charged mitochondria. Unlike receptor:ligand approaches that rely upon stochastic Brownian motion for the ligand to approach the receptor, the mitochondrial localization signal of some embodiments is drawn to mitochondria because of charge. Therefore, in some embodiments, the mitochondrial targeting agent is a protein transduction domain including but not limited to the protein transduction domains discussed in detail above.

Mitochondrial targeting agents also include short peptide sequences (Yousif, et al., Chembiochem., 10(13):2131 (2009), for example mitochondrial transporters-synthetic cell-permeable peptides, also known as mitochondria-penetrating peptides (MPPs), that are able to enter mitochondria. MPPs are typically cationic, but also lipophilic; this combination of characteristics facilitates permeation of the hydrophobic mitochondrial membrane. For example, MPPs can include alternating cationic and hydrophobic residues (Horton, et al., Chem Biol., 15(4):375-82 (2008)). Some MPPs include delocalized lipophilic cations (DLCs) in the peptide sequence instead of, or in addition to natural cationic amino acids (Kelley, et al., Pharm. Res., 2011 Aug. 11 [Epub ahead of print]). Other variants can be based on an oligomeric carbohydrate scaffold, for example attaching guanidinium moieties due to their delocalized cationic form (Yousif, et al., Chembiochem., 10(13):2131 (2009).

Mitochondrial targeting agents also include mitochondrial localization signals or mitochondrial targeting signals. Many mitochondrial proteins are synthesized as cytosolic precursor proteins containing a leader sequence, also known as a presequence, or peptide signal sequence. Typically, cytosolic chaperones deliver the precursor protein to mitochondrial receptors and the General Import Pore (GIP) (Receptors and GIP are collectively known as Translocase of Outer Membrane or TOM) at the outer membrane. Typically, the precursor protein is translocated through TOM, and the intermembrane space by small TIMs to the TIM23 or 22 (Translocase of Inner Membrane) at the inner membrane. Within the mitochondrial matrix the targeting sequence is cleaved off by mtHsp70.

As discussed above, in order to enter the mitochondria, a protein generally must interact with the mitochondrial import machinery, consisting of the Tim and Tom complexes (Translocase of the Inner/Outer Mitochondrial Membrane). With regard to the mitochondrial targeting signal, the positive charge draws the linked protein to the complexes and continues to draw the protein into the mitochondria. The Tim and Tom complexes allow the proteins to cross the membranes. Accordingly, one embodiment of the present disclosure delivers compositions of the present disclosure to the inner mitochondrial space utilizing a positively charged targeting signal and the mitochondrial import machinery. In another embodiment, PTD-linked compounds containing a mitochondrial localization signal do not seem to utilize the TOM/TIM complex for entry into the mitochondrial matrix, see Del Gaizo et al. Mol Genet Metab. 80(1-2):170-80 (2003). The N-terminal region of the proteins can be used to target molecules to the mitochondrion. The sequences are known in the art, see for example, U.S. Pat. No. 8,039,587, which is specifically incorporated by reference herein in its entirety. The identification of the specific sequences necessary for translocation of a linked compound into a mitochondrion can be determined using predictive software known to those skilled in the art, including the tools located at http://ihg.gsf.de/ihg/mitoprot.html. Using the software the predicted sequence from Etfa that can be used to target the disclosed composition is

(SEQ ID NO: 16)
MFRAAAPGQL RRAASLLRF.

mitochondrial targeting signal from Dld is

(SEQ ID NO: 17)
MQSWSRVYCS LAKRGHFNRI
SHGLQGLSAV PLRTY.

In certain embodiments, the mitochondrial targeting agent is the mitochondrial localization signal of a mangano-superoxide dismutase (also referred to herein as “SOD2” and “Mn-SOD” and “superoxide dismutase (Mn)) precursor protein. Several mitochondrial localization signals for SOD2 are known in the art. In some embodiments the mitochondrial targeting signal includes the amino acid sequence

(SEQ ID NO: 18)
MLSRAVCGTS RQLPPVLGYL GSRQ

or SEQ ID NO: 18 without the N-terminal methionine

(SEQ ID NO: 19)
LSRAVCGTSR QLPPVLGYLG SRQ.

In another embodiment the mitochondrial targeting signal includes the amino acid sequence

(SEQ ID NO: 20)
MLSRAVCGTS RQLAPVLGYL GSRQ;

or SEQ ID NO:20 without the N-terminal methionine

(SEQ ID NO: 21)
LSRAVCGTSR QLAPVLGYLG SRQ.

In some embodiments, the composition is preferentially delivered to the mitochondrial using a mitochondrial delivery vehicle, such as a lipid raft, mitochondrially targeted nanoparticle, or mitochondriotropic liposome. In such cases, one or more polynucleotide-binding polypeptides can be associated with, encapsulated within, dispersed in or on, or covalently attached to the mitochondrial delivery vehicle.

In certain embodiments, polynucleotide-binding polypeptides are encapsulated, coupled to, or otherwise associated with mitochondriotropic liposomes. Mitochondriotrophic liposomes are cationic liposomes that can be used to deliver an encapsulated agent to the mitochondria of a cell. Mitochondriotropic liposomes are known in the art. See, for example, U.S. Patent Application Publication No. US 2008/0095834 to Weissig, et al, which is specifically incorporated herein by reference in its entirety. Mitochondriotropic liposomes are liposomes which contain a hydrophobized amphiphilic delocalized cation, such as a triphenylphosphonium or a quinolinium moiety, incorporated into or conjugate to the lipid membrane of the liposome. As a result, the liposomes can be used to deliver compounds incorporated within them to the mitochondria.

In other embodiments, polynucleotide-binding polypeptides are encapsulated within, dispersed in, associated with, or conjugated to a nanoparticle functionalized with one or more mitochondrial targeting agents. For example, the nanoparticle may contain one or be functionalized with one or more lipophilic cations or polypeptide targeting agents.

The nanoparticles may be formed from one or more polymers, copolymers, or polymer blends. In some embodiments, the one or more polymers, copolymers, or polymer blends are biodegradable. Examples of suitable polymers include, but are not limited to, polyhydroxyacids such as poly(lactic acid), poly(glycolic acid), and poly(lactic acid-co-glycolic acids); polycaprolactones; poly(orthoesters); polyanhydrides; poly(phosphazenes); poly(hydroxyalkanoates); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxy alkylmethacrylamide), poly(hydroxyalkylmethacrylate), poly(saccharides), poly(vinyl alcohol), as well as blends and copolymers thereof. Techniques for preparing suitable polymeric nanoparticles are known in the art, and include solvent evaporation, hot melt particle formation, solvent removal, spray drying, phase inversion, coacervation, and low temperature casting. In some cases, the mitochondrial targeting agents are polypeptides that are covalently linked to the surface of the nanoparticle after particle formulation. In other cases, the mitochondrial targeting agents are lipophilic cations that are covalently bound to the particle surface. In some cases, a cationic polymer is incorporated into the particle to target the particle to the mitochondrion.

Polynucleotide-binding polypeptides can also be targeted to the mitochondria using lipid rafts or other synthetic vesicle compositions. See, for example, U.S. Patent Application Publication No. US 2007/0275924 to Khan, et al. which is specifically incorporated herein by reference in its entirety. The lipid raft compositions can include cholesterol, and one or more lipids selected from the group consisting of sphingomylein, gangliosides, phosphatidylethanolamine, phosphatidylserine, phosphatidylcholine, phosphatidylinositol, and a mitochondrial targeting agent. In certain embodiments, a polypeptide targeting agent is inserted into the lipid raft to target the raft to the mitochondria. The lipid rafts can be prepared and loaded with one or more polynucleotide-binding polypeptides using methods known in the art. See, for example, U.S. Pat. No. 6,156,337 to Barenholz, et al.

A preferred polynucleotide-binding polypeptide that targets mitochondria has at least 80, 85, 90, 95, 99 or 100 percent sequence identity to

(SEQ ID NO: 22)
MARRRRRRRR RRRMAFLRSM WGVLSALGRS GAELCTGCGS
RLRSPFSFVY LPRWFSSVLA SCPKKPVSSY LRFSKEQLPI
FKAQNPDAKT TELIRRIAQR WRELPDSKKK IYQDAYRAEW
QVYKEEISRF KEQLTPSQIM SLEKEIMDKH LKRKAMTKKK
ELTLLGKPKR PRSAYNVYVA ERFQEAKGDS PQEKLKTVKE
NWKNLSDSEK ELYIQHAKED ETRYHNEMKS WEEQMIEVGR
KDLLRRTIKK QRKYGAEEC,
or
SEQ ID NO: 22 without the N-terminal
methionine
(SEQ ID NO: 23)
ARRRRRRRRR RRMAFLRSMW GVLSALGRSG AELCTGCGSR
LRSPFSFVYL PRWFSSVLAS CPKKPVSSYL RFSKEQLPIF
KAQNPDAKTT ELIRRIAQRW RELPDSKKKI YQDAYRAEWQ
VYKEEISRFK EQLTPSQIMS LEKEIMDKHL KRKAMTKKKE
LTLLGKPKRP RSAYNVYVAE RFQEAKGDSP QEKLKTVKEN
WKNLSDSEKE LYIQHAKEDE TRYHNEMKSW EEQMIEVGRK
DLLRRTIKKQ RKYGAEEC.
Another embodiment provides a nucleic
acid encoding the polypeptide according
to SEQ ID NO: 22 is
(SEQ ID NO: 24)
ATGGCGCGTC GTCGTCGTCG TCGTCGTCGT CGTCGTCGTA
TGGCGTTTCT CCGAAGCATG TGGGGCGTGC TGAGTGCCCT
GGGAAGGTCT GGAGCAGAGC TGTGCACCGG CTGTGGAAGT
CGACTGCGCT CCCCCTTCAG TTTTGTGTAT TTACCGAGGT
GGTTTTCATC TGTCTTGGCA AGTTGTCCAA AGAAACCTGT
AAGTTCTTAC CTTCGATTTT CTAAAGAACA ACTACCCATA
TTTAAAGCTC AGAACCCAGA TGCAAAAACT ACAGAACTAA
TTAGAAGAAT TGCCCAGCGT TGGAGGGAAC TTCCTGATTC
AAAGAAAAAA ATATATCAAG ATGCTTATAG GGCGGAGTGG
CAGGTATATA AAGAAGAGAT AAGCAGATTT AAAGAACAGC
TAACTCCAAG TCAGATTATG TCTTTGGAAA AAGAAATCAT
GGACAAACAT TTAAAAAGGA AAGCTATGAC AAAAAAAAAA
GAGTTAACAC TGCTTGGAAA ACCAAAAAGA CCTCGTTCAG
CTTATAACGT TTATGTAGCT GAAAGATTCC AAGAAGCTAA
GGGTGATTCA CCGCAGGAAA AGCTGAAGAC TGTAAAGGAA
AACTGGAAAA ATCTGTCTGA CTCTGAAAAG GAATTATATA
TTCAGCATGC TAAAGAGGAC GAAACTCGTT ATCATAATGA
AATGAAGTCT TGGGAAGAAC AAATGATTGA AGTTGGACGA
AAGGATCTTC TACGTCGCAC AATAAAGAAA CAACGAAAAT
ATGGTGCTGA GGAGTGTTAA.

The sequence encoding the protein transduction domain is underlined, and the sequence encoding the mitochondrial localization signal is double underline. Still another embodiment provides a nucleic acid having at least 80, 85, 90, 95, 99 or more percent sequence identity to SEQ ID NO:24

Another preferred polynucleotide-binding polypeptides that targets mitochondria has at least 80, 85, 90, 95, 97, 99, or 100 percent sequence identity to

(SEQ ID NO: 25)
MRRRRRRRRR RRGEGDIMGE WGNEIFGAIA GFLGGEMLSR
AVCGTSRQLP PVLGYLGSRQ SSVLASCPKK PVSSYLRFSK
EQLPIFKAQN PDAKTTELIR RIAQRWRELP DSKKKIYQDA
YRAEWQVYKE EISRFKEQLT PSQIMSLEKE IMDKHLKRKA
MTKKKELTLL GKPKRPRSAY NVYVAERFQE AKGDSPQEKL
KTVKENWKNL SDSEKELYIQ HAKEDETRYH NEMKSWEEQM
IEVGRKDLLR RTIKKQRKYG AEEC,
or
SEQ ID NO: 25 without the N-terminal
methionine
(SEQ ID NO: 26)
RRRRRRRRRR RGEGDIMGEW GNEIFGAIAG FLGGEMLSRA
VCGTSRQLPP VLGYLGSRQS SVLASCPKKP VSSYLRFSKE
QLPIFKAQNP DAKTTELIRR IAQRWRELPD SKKKIYQDAY
RAEWQVYKEE ISRFKEQLTP SQIMSLEKEI MDKHLKRKAM
TKKKELTLLG KPKRPRSAYN VYVAERFQEA KGDSPQEKLK
TVKENWKNLS DSEKELYIQH AKEDETRYHN EMKSWEEQMI
EVGRKDLLRR TIKKQRKYGA EEC

In another embodiment, the recombinant polypeptide is encoded by a nucleic acid having at least 80, 85, 90, 95, 97, 99, or 100% sequence identity to

(SEQ ID NO: 27)
ATGCGGCGAC GCAGACGTCG TCGTCGGCGG CGTCGCGGCG
AGGGTGATAT TATGGGTGAA TGGGGGAACG AAATTTTCGG
AGCGATCGCT GGTTTTCTCG GTGGAGAAAT GTTATCACGC
GCGGTATGTG GCACCAGCAG GCAGCTGCCT CCAGTCCTTG
GCTATCTGGG TTCCCGCCAG TCATCGGTGT TAGCATCATG
TCCGAAAAAA CCTGTCTCGT CGTACCTGCG CTTCTCCAAA
GAGCAGCTGC CGATTTTTAA AGCGCAAAAT CCGGATGCTA
AAACGACTGA ACTGATTCGC CGCATTGCAC AACGCTGGCG
CGAACTCCCG GACAGTAAAA AAAAAATTTA TCAGGACGCC
TATCGGGCTG AATGGCAGGT CTATAAAGAG GAGATCTCAC
GCTTCAAAGA ACAATTAACC CCGAGTCAAA TAATGTCTCT
GGAAAAAGAA ATCATGGATA AACACTTAAA ACGAAAGGCG
ATGACGAAGA AAAAAGAACT GACCCTGCTA GGTAAACCTA
AGCGTCCGCG CTCTGCGTAT AATGTGTACG TGGCAGAACG
TTTTCAGGAG GCCAAAGGGG ATTCTCCGCA AGAAAAACTG
AAGACCGTCA AAGAAAATTG GAAAAACCTG TCTGATAGCG
AAAAAGAACT GTACATTCAG CACGCTAAAG AAGATGAGAC
GCGGTATCAC AACGAAATGA AATCTTGGGA AGAGCAGATG
ATCGAGGTCG GTCGGAAGGA TCTTCTCCGT CGAACCATCA
AAAAACAGCG TAAATATGGA GCAGAAGAGT GCTGA.

Preferably the mitochondrial targeting signal, domain, or agent does not permanently damage the mitochondrion, for example the mitochondrial membrane, or otherwise impair mitochondrial function.

b. Nuclear Localization Signals

The polynucleotide-binding polypeptides disclosed herein can include one or more nuclear localization signals. Nuclear localization signals (NLS) or domains are known in the art and include for example, SV 40 T antigen or a fragment thereof, such as

(SEQ ID NO: 40)
PKKKRKV.

The NLS can be simple cationic sequences of about 4 to about 8 amino acids, or can be bipartite having two interdependent positively charged clusters separated by a mutation resistant linker region of about 10-12 amino acids. Additional representative NLS include but are not limited to

(SEQ ID NO: 28)
GKKRSKV;
(SEQ ID NO: 29)
KSRKRKL;
(SEQ ID NO: 30)
KRPAATKKAG QAKKKKLDK;
(SEQ ID NO: 31)
RKKRKTEEES PLKDKAKKSK;
(SEQ ID NO: 32)
KDCVMNKHHR NRCQYCRLQR;
(SEQ ID NO: 33)
PAAKRVKLD;
and
(SEQ ID NO: 34)
KKYENVVIKR SPRKRGRPRK.

ii. Cell Targeting

The proteins of interest disclosed herein can be modified to target a specific cell type or population of cells.

For example, the proteins of interest can be modified with galactosyl-terminating macromolecules to target the polypeptide of interest to the liver or to liver cells. The modified polypeptide of interest selectively enters hepatocytes after interaction of the carrier galactose residues with the asialoglycoprotein receptor present in large amounts and high affinity only on these cells.

In one embodiment, the targeting signal binds to its ligand or receptor which is located on the surface of a target cell such as to bring the composition and cell membranes sufficiently close to each other to allow penetration of the composition into the cell.

In a preferred embodiment, the targeting molecule is selected from the group consisting of an antibody or antigen binding fragment thereof, an antibody domain, an antigen, a T-cell receptor, a cell surface receptor, a cell surface adhesion molecule, a major histocompatibility locus protein, a viral envelope protein and a peptide selected by phage display that binds specifically to a defined cell.

Targeting a polypeptide of interest to specific cells can be accomplished by modifying the polypeptide of interest to express specific cell and tissue targeting signals. These sequences target specific cells and tissues. In some embodiments the interaction of the targeting signal with the cell does not occur through a traditional receptor:ligand interaction. The eukaryotic cell comprises a number of distinct cell surface molecules. The structure and function of each molecule can be specific to the origin, expression, character and structure of the cell. Determining the unique cell surface complement of molecules of a specific cell type can be determined using techniques well known in the art.

One skilled in the art will appreciate that the tropism of the proteins of interest described can be altered by changing the targeting signal. In one specific embodiment, compositions are provided that enable the addition of cell surface antigen specific antibodies to the composition for targeting the delivery of polynucleotide-binding polypeptide. Exemplary cell surface antigens are disclosed in Wagner et al., Adv Gen, 53:333-354 (2005) which is specifically incorporated by reference herein in its entirety.

It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used a targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest

a. Brain Targeting

In one embodiment, the targeting signal is directed to cells of the nervous system, including the brain and peripheral nervous system. Cells in the brain include several types and states and possess unique cell surface molecules specific for the type. Furthermore, cell types and states can be further characterized and grouped by the presentation of common cell surface molecules.

In one embodiment, the targeting signal is directed to specific neurotransmitter receptors expressed on the surface of cells of the nervous system. The distribution of neurotransmitter receptors is well known in the art and one so skilled can direct the compositions described by using neurotransmitter receptor specific antibodies as targeting signals. Furthermore, given the tropism of neurotransmitters for their receptors, in one embodiment the targeting signal consists of a neurotransmitter or ligand capable of specifically binding to a neurotransmitter receptor.

In one embodiment, the targeting signal is specific to cells of the nervous system which may include astrocytes, microglia, neurons, oligodendrites and Schwann cells. These cells can be further divided by their function, location, shape, neurotransmitter class and pathological state. Cells of the nervous system can also be identified by their state of differentiation, for example stem cells. Exemplary markers specific for these cell types and states are well known in the art and include, but are not limited to CD133 and Neurosphere.

b. Muscle Targeting

In one embodiment, the targeting signal is directed to cells of the musculoskeletal system. Muscle cells include several types and possess unique cell surface molecules specific for the type and state. Furthermore, cell types and states can be further characterized and grouped by the presentation of common cell surface molecules.

In one embodiment, the targeting signal is directed to specific neurotransmitter receptors expressed on the surface of muscle cells. The distribution of neurotransmitter receptors is well known in the art and one so skilled can direct the compositions described by using neurotransmitter receptor specific antibodies as targeting signals. Furthermore, given the tropism of neurotransmitters for their receptors, in one embodiment the targeting signal consists of a neurotransmitter. Exemplary neurotransmitters expressed on muscle cells that can be targeted include but are not limited to acetycholine and norepinephrine,

In one embodiment, the targeting signal is specific to muscle cells which consist of two major groupings, Type I and Type II. These cells can be further divided by their function, location, shape, myoglobin content and pathological state. Muscle cells can also be identified by their state of differentiation, for example muscle stem cells. Exemplary markers specific for these cell types and states are well known in the art include, but are not limited to MyoD, Pax7 and MR4.

c. Tumor Targeting

In one embodiment, the targeting signal is used to selectively target tumor cells. Tumor cells express cell surface markers which may be selectively expressed by tumor cells. Other cell surface markers are preferentially expressed by tumor cells but can be expressed by non-tumor cells in certain circumstances. Agents that specifically recognize the tumor cell surface markers can be used to target the compositions to the tumor cells.

Exemplary tumor specific cell surface markers include, but are not limited to, alfa-fetoprotein (AFP), C-reactive protein (CRP), cancer antigen-50 (CA-50), cancer antigen-125 (CA-125) associated with ovarian cancer, cancer antigen 15-3 (CA15-3) associated with breast cancer, cancer antigen-19 (CA-19) and cancer antigen-242 associated with gastrointestinal cancers, carcinoembryonic antigen (CEA), carcinoma associated antigen (CAA), chromogranin A, epithelial mucin antigen (MC5), human epithelium specific antigen (HEA), Lewis(a)antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdr1 gene product), multidrug-resistance-related antigen, p170, multidrug-resistance-related antigen, prostate specific antigen (PSA), CD56, and NCAM.

In one embodiment, the targeting signal is an antibody specific for a tumor cell surface marker.

d. Antibodies

Another embodiment provides an antibody or antigen binding fragment thereof bound to the disclosed proteins of interest acting as the targeting signal. The antibodies or antigen binding fragment thereof are useful for directing the vector to a cell type or cell state. In one embodiment, the polypeptide of interest possesses an antibody binding domain, for example from proteins known to bind antibodies such as Protein A and Protein G from Staphylococcus aureus. For example, some embodiments the polypeptide includes the amino acids sequence

(SEQ ID NO: 41)
HDEAQQNAFY QVLNMPNLNA DQRNGFIQSL KDDPSQSANV
LGEAHDEAQQ NAFYQVLNMP NLNADQRNGF IQSLKDDPSQ
SANVLGEA
or
(SEQ ID NO: 42)
HDEAQQNAFY QVLNMPNLNA DQRNGFIQSL KDDPSQSANV
LGEAHDEAQQ NAFYQVLNMP NLNADQRNGF IQSLKDDPSQ
SANVLGEAGE G,

both of which include the tandem domain B of Protein A.

In a preferred embodiment, the polynucleotide-binding protein has at least 80, 85, 90, 95, 99, or 100 percent sequence identity to

(SEQ ID NO: 43)
MRRRRRRRRR RRGEGDIMGE WGNEIFGAIA GFLGGEHDEA
QQNAFYQVLN MPNLNADQRN GFIQSLKDDP SQSANVLGEA
HDEAQQNAFY QVLNMPNLNA DQRNGFIQSL KDDPSQSANV
LGEAGEGSSV LASCPKKPVS SYLRFSKEQL PIFKAQNPDA
KTTELIRRIA QRWRELPDSK KKIYQDAYRA EWQVYKEEIS
RFKEQLTPSQ IMSLEKEIMD KHLKRKAMTK KKELTLLGKP
KRPRSAYNVY VAERFQEAKG DSPQEKLKTV KENWKNLSDS
EKELYIQHAK EDETRYHNEM KSWEEQMIEV GRKDLLRRTI
KKQRKYGAEE C,
or
SEQ ID NO: 43 without the N-terminal
methionine
(SEQ ID NO: 44)
RRRRRRRRRR RGEGDIMGEW GNEIFGAIAG FLGGEHDEAQ
QNAFYQVLNM PNLNADQRNG FIQSLKDDPS QSANVLGEAH
DEAQQNAFYQ VLNMPNLNAD QRNGFIQSLK DDPSQSANVL
GEAGEGSSVL ASCPKKPVSS YLRFSKEQLP IFKAQNPDAK
TTELIRRIAQ RWRELPDSKK KIYQDAYRAE WQVYKEEISR
FKEQLTPSQI MSLEKEIMDK HLKRKAMTKK KELTLLGKPK
RPRSAYNVYV AERFQEAKGD SPQEKLKTVK ENWKNLSDSE
KELYIQHAKE DETRYHNEMK SWEEQMIEVG RKDLLRRTIK
KQRKYGAEEC.

Other domains known to bind antibodies are known in the art and can be substituted. In certain embodiments, the antibody is polyclonal, monoclonal, linear, humanized, chimeric or a fragment thereof. Representative antibody fragments are those fragments that bind the antibody binding portion of the non-viral vector and include Fab, Fab′, F(ab′), Fv diabodies, linear antibodies, single chain antibodies and bispecific antibodies known in the art.

In some embodiments, the targeting domain includes all or part of an antibody that directs the vector to the desired target cell type or cell state. Antibodies can be monoclonal or polyclonal, but are preferably monoclonal. For human gene therapy purposes, antibodies are derived from human genes and are specific for cell surface markers, and are produced to reduce potential immunogenicity to a human host as is known in the art. For example, transgenic mice which contain the entire human immunoglobulin gene cluster are capable of producing “human” antibodies can be utilized. In one embodiment, fragments of such human antibodies are employed as targeting signals. In a preferred embodiment, single chain antibodies modeled on human antibodies are prepared in prokaryotic culture.

In preferred embodiments the polypeptide of interest is itself a fusion protein. The fusion protein can include, for example, a polynucleotide-binding polypeptide, a protein transduction domain, and optionally one or more targeting signals. A preferred polypeptide of interest is SEQ ID NO:26. Other exemplary fusion proteins containing a mitochondrial transcription factor polypeptide that are suitable for use as a polypeptide of interest are disclosed in U.S. Pat. No. 8,039,587 and U.S. Patent Publication Nos. 2009/0123468, 2009/0208478, 2009/0227655, and 2006/0211647 all of which are specifically incorporated by reference in their entireties.

4. Additional Sequences

The fusion protein can optionally include additional sequences or moieties, including, but not limited to linkers and purification tags.

In a preferred embodiment the purification tag is a polypeptide. Polypeptide purification tags are known in the art and include, but are not limited to His tags which typically include six or more, typically consecutive, histidine residues; FLAG tags, which typically include the sequence

(SEQ ID NO: 35)
DYKDDDDK;

hemagglutinin (SA) for example,

(SEQ ID NO: 36)
YPYDVP;

MYC tag for example

(SEQ ID NO: 37)
ILKKATAYIL
or
(SEQ ID NO: 38)
EQKLISEEDL.

Methods of using purification tags to facilitate protein purification are known in the art and include, for example, a chromatography step wherein the tag reversibly binds to a chromatography resin.

Purifications tags can be N-terminal or C-terminal to the fusion protein. The purification tags N-terminal to the fusion protein are typically separated from the polypeptide of interest at the time of the cleavage in vivo. Therefore, purification tags N-terminal to the fusion protein can be used to remove the fusion protein from a cellular lysate following expression and extraction of the expression or solubility enhancing amino acid sequence, but cannot be used to remove the polypeptide of interest. Purification tags C-terminal to the fusion protein can be used to remove the polypeptide of interest from a cellular lysate following expression of the fusion protein, but cannot be used to remove the expression or solubility enhancing amino acid sequence. Purification tags that are C-terminal to the expression or solubility enhancing amino acid sequence can be N-terminal to, C-terminal to, or incorporated within the sequence of the polypeptide of interest.

In some embodiments, to fusion protein includes one or more linkers or spacers. In some embodiments linker or spacer is one or more polypeptides. In some embodiments, the linker includes a glycine-glutamic acid di-amino acid sequence. The linkers can be used to link or connect two domains, regions, or sequences of the fusion protein.

5. Protein Expression

Molecular biology techniques have developed so that therapeutic proteins can be genetically engineered to be expressed by microorganisms. The gram negative bacterium, Escherichia coli, is a versatile and valuable organism for the expression of therapeutic proteins. Although many proteins with therapeutic or commercial uses can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. Some of these problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some embodiments, the compositions disclosed herein include expression or solubility enhancing amino acid sequence. In some embodiments, the expression or solubility enhancing amino acid sequence is cleaved prior administration of the composition to a subject in need thereof. The expression or solubility enhancing amino acid sequence can be cleaved in the recombinant expression system, or after the expressed protein in purified. In some embodiments, the expression or solubility enhancing is a ULP1 or SUMO sequence. Recombinant protein expression systems that incorporate the SUMO protein (“SUMO fusion systems”) have been shown to increase efficiency and reduce defective expression of recombinant proteins in E. coli., see for example Malakhov, et al., J. Struct. Funct. Genomics, 5: 75-86 (2004), U.S. Pat. No. 7,060,461, and U.S. Pat. No. 6,872,551. SUMO fusion systems enhance expression and solubility of certain proteins, including severe acute respiratory syndrome coronavirus (SARS-CoV) 3CL protease, nucleocapsid, and membrane proteins (Zuo et al., J. Struct. Funct. Genomics, 6:103-111 (2005)).

B. Combination Therapies

The compositions disclosed herein can be used alone, or in combination with one or more additional therapeutic agents. “In combination” refers to simultaneous or in tandem administration as a single formulation or in multiple formulations.

The compositions disclosed herein and the second therapeutic can also be administered consecutively. Consecutive administration refers to separate, individual formulations for each composition. The compositions can be administered in any order. The term consecutive administration refers to administration of one composition and then at least 30 minutes later administering the other composition. The consecutive administration can be at least 30 minutes, 45 minutes, 1 hour, 2 hours, 3 hours, 4 hours, 5 hours, 10 hours, 12 hours, 18 hours, 1 day, 2 days, 3 days, 4 days, 5 days, 6 days, 7 days, 10 days, 14 days or 30 days from the administration of the first composition.

1. Immunotherapeutics

In some embodiments a second therapeutic is an immunotherapeutic that is optionally directed to a specific cell type. For example, the tumor cell can bear some marker that is amenable to targeting, i.e., is not present on the majority of other cells. Many suitable tumor markers exist and are known in the art. Common tumor markers include, but are not limited to, alfa-fetoprotein (AFP), C-reactive protein (CRP), cancer antigen-50 (CA-50), cancer antigen-125 (CA-125) associated with ovarian cancer, cancer antigen 15-3 (CA15-3) associated with breast cancer, cancer antigen-19 (CA-19) and cancer antigen-242 associated with gastrointestinal cancers, carcinoembryonic antigen (CEA), carcinoma associated antigen (CAA), chromogranin A, epithelial mucin antigen (MC5), human epithelium specific antigen (HEA), Lewis(a)antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdr1 gene product), multidrug-resistance-related antigen, p170, multidrug-resistance-related antigen, prostate specific antigen (PSA), CD56, NCAM, carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to induce anticancer effects with immune stimulatory effects.

Examples of immune stimulating molecules include, but are not limited to, cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects.

Examples of immunotherapies are discussed in more detail below, and include, but are not limited to immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons, and; IL-1, GM-CSF and TNF) gene therapy (e.g., TNF, IL-1, IL-2, p53) (U.S. Pat. No. 5,830,880 and U.S. Pat. No. 5,846,945), monoclonal antibodies (e.g., anti-ganglioside GM2, anti-HER-2, anti-p185) (U.S. Pat. No. 5,824,311) and oncolytic virus or virotherapy.

a. Peptides and Proteins

In some embodiments, the additional therapeutic is a peptide or protein. The peptides or portions thereof used as immunotherapies can not only bind to an MHC Class I molecule, but also can stimulate CD8⁺ T cells, e.g., cytotoxic T lymphocytes (CTL). By “stimulate” in the context of T cells is meant activating intracellular signaling pathways in a T cell through the antigen-specific T cell receptor (TCR) expressed on that T cell, which activation leads to one or more T cell responses, such as T cell proliferation, cytolytic activity, and cytokine production, e.g., IFN-γ. Preferably, the T cells are stimulated by the immunotherapeutic peptides to kill or lyse a target cell, which presents the peptide recognized by the TCR of the T cell. In preferred embodiments, the target cell is a tumor cell. Also, in some instances, it is preferable for the peptides to stimulate CD4⁺ T cells, in addition to CD8⁺ T cells. Stimulation of CD4⁺ T cells by the immunogenic peptide aids in a B cell mediated immune response, which includes the production of antibodies.

Examples of peptides for use as an immunotherapy are disclosed in U.S. Publication Nos. 20110081352 and 20090280511 which are specifically incorporated by reference herein in their entireties.

In some embodiments, the peptide or protein is a chemokine and cytokine, for example a recombinant cytokine which enhances or stimulates the humoral or cellular immune response. Cytokines include but are not limited to, interleukins, lymphokines, monokines, interferons, colony stimulating factors and chemokines. Examples of specific cytokine that may be used include, but are not limited to, IL-1, TNF, IL-2, IFN-γ, IL-4, IL-6, IL-7, IL-8, IL-12 and GM-CSF. The recombinant proteins may comprise all or part of one or more cytokines or chemokines.

The recombinant proteins can be fusion proteins. The fusion proteins can interact with two or more different cells. For instance, part of the fusion protein can interact with the natural killer activating receptor on a tumor cell and the other part of the protein can interact with the T cell receptor on a T cell (Zhang et al., Cancer Res., 71:2066-76, (2011)). This interaction brings the two cells in proximity and stimulates the T cell to kill the tumor cell. The fusion proteins can be a cancer or virus antigen binding protein and the Fc region of immunoglobulin. The presence of the Fc region aids in the clearance of anything bound to the fusion protein.

Common protein immunotherapies are any protein that stimulates or acts as a co-stimulatory molecule for immune cells. For example, an immunotherapy can be any protein that utilizes the B7/CD28 pathway. The recombinant protein can include the full protein or extracellular domain of B7, CD137-L, CD134-L, GITR-L or CD40. The recombinant protein could be a fusion wherein it includes a combination of co-stimulatory molecules.

In some embodiments, the formulation for administration includes the protein or peptide. In some embodiments, the formulation for administration includes a polynucleotide encoding the peptide or protein such that the peptide or protein is translated in vivo. For example, in some embodiments, the formulation for administration includes a vector encoding the peptide or protein.

DNA vectors can include the nucleic acid sequences that encode one or more of the proteins. Most genes can be incorporated into an expression vector. DNA vectors can include the nucleic acid sequence that encodes the bacterial toxin ETA along with a nucleic acid sequence that encodes a tumor antigen (Hung et al., Cancer Res. 61:3698-3703, 2001). ETA allows translocation of the protein to the cytosol. The presence of the protein in the cytosol enhances MHC I presentation.

Viral vectors can also be used to deliver polynucleotides. The viral vectors can be derived from but are not limited to adenovirus, adeno-associated virus, retrovirus, lentivirus, vaccinia virus, poxvirus, herpesvirus, or RNA viruses. Viral vectors can carry the gene for any of the proteins or peptides disclosed herein. Viral vectors can carry genes for, but are not limited to, cytokines, tumor or viral antigens or pro-apoptotic molecules.

b. Therapeutic Nucleic Acids

In some embodiments, the additional therapeutic is a nucleic acid. For example, CpG DNA is immunostimulatory. CpG oligonucleotides can stimulate cytokine production, natural killer cell activity, and B cell proliferation. Examples of immunostimulatory DNA can be found in U.S. Publication 20110081366.

c. Antibodies

In some embodiments, the additional therapeutic is an antibody. Antibodies can be used to mark the target cell, such as a cancer cell, so that the immune system can easily find the cancer cells. The monoclonal antibody drug rituximab is an example. Rituximab binds to CD20 found on the surface of B cells allowing for the antibody-labeled B cells to be destroyed by the immune system. Destruction of antibody coated cells can occur via antibody-dependent cell-mediated cytotoxicity (ADCC). Briefly, antibodies bound on the surface of a cancer cell or on the surface of a cell haboring a virus can act as a label or marker. These labeled cells can then be bound by natural killer (NK) cells, which recognize the Fc region, or constant region, of the antibody. The interaction of the NK cells with the Fc region of the antibody initiates a signaling pathway in the NK cells resulting in the activated NK cell destroying the antibody bound cell (i.e. the cancer cell or virus infected cell).

Antibodies can be used to block signals required for cell growth. Growth factors bind to growth factor receptors on the surface of cells signaling the cells to grow. And increase in the growth factor receptors on the surface of cancer cells causes them to grow faster than normal cells. Cetuximab and panitumumab are monoclonal antibodies for treating colon cancer as well as head and neck cancers. They bind to epidermal growth factor (EGF) receptors on the surface of cells and prevent EGF from binding to the receptors. The blocking of the EGF signaling pathway can slow or stop cancer cell growth. These antibodies led to the degradation of EGF receptors, which ultimately lead to the presentation of EGF receptor peptides on MHC Ia molecules. The presentation of these peptides on MHC Ia lead to an increase in cytotoxic T cells specific to these peptides (Andrade Filho et al., J Immunother, 33:83-91 (2010)).

Another common antibody therapeutic which causes internalization and degradation of its receptor ligand is trastuzumab. The internalization and degradation leads to enhanced MHC I presentation of receptor-derived peptides and therefore the humanized antibody, trastuzumab, indirectly induces a cytotoxic T cell response to tumor cells (Milano et al., PLoS One 5:e12424, 2010; Kono et al., Clin Cancer Res. 10:2538-44, 2004).

Monoclonal Antibodies may block cell signal transduction not only by blocking the ligand to bind to its receptor, but inhibiting the dimerization of said receptor and in such a way block the signaling cascade, as in the case with Pertuzumab, a dimerization inhibitor of the human EGF receptor HER2. Other receptors may be inhibited by similar mechanisms.

Similar to blocking cell growth, antibodies can block signals required for attracting blood vessels to the cancer cells. Bevacizumab is an example of a monoclonal antibody that targets vascular endothelial growth factor (VEGF) and prevents the growth of new blood vessels and can cause existing blood vessels to shrink.

Another blocking mechanism employed by therapeutic antibodies is the blocking of molecules that send inhibitory signals to immune cells. For example, CTLA-4 acts a second signal on helper T cells (CD4+ cells). Co-stimulation through the T cell receptor and CTLA-4 results in inhibition of normal T cell activation. This inhibition can prevent helper T cells from performing their normal immune functions. Antibodies such as ipilimumab and tremelimumab target and block CTLA-4. Blocking the CTLA-4 inhibitory signaling molecule allows for proper activation of the immune system for fighting cancer cells and virus infected cells.

As mentioned above, antibodies can be coupled to an agent such as a radioactive isotope. For instance, Ibritumomab treats non-Hodgkin's lymphoma by using a monoclonal antibody to cancerous blood cells to deliver a radioactive particle.

It may be favorable to administer more than one monoclonal antibody directed against two different antigens or even antibodies with multiple antigen specificity. For instance, bispecific antibodies can recognize a tumor antigen and an antigen which promotes the release of a cytokine such as IL-1 or TNF alpha. Treatment protocols also may include administration of lymphokines or other immune enhancers as described by Bajorin et al. (Comparison of criteria for assigning germ cell tumor patients to “good risk” and “poor risk” studies J. Clin. Oncol., 6(5):786-92, 1988). The development of human monoclonal antibodies is described in further detail elsewhere in the specification.

Antibodies can be used alone or coupled with toxins, chemotherapeutic agents or radioactive isotopes. The antibodies can be monoclonal antibodies. Preferably, human monoclonal antibodies are employed, as they produce few or no side effects in the patient.

d. Oncolytic Viruses

In some embodiments, the additional therapeutic is an oncolytic virus. Viruses may have native properties, or may be engineered, to lyse cancer cells. The Reovirus replicates predominantly in cells with an activated Ras pathway, and leaves cells with non-elevated Ras activity unaffected. Ras, and other members of the Ras signal transduction cascade, are commonly oncogenes, and is activated in a wide variety of cancers. Reolysin® is an example of a pharmaceutical approach based on these premises. In the case of engineered oncolytic viruses, they may be made to contain chemokines or cytokines, to enhance any immune response to tumors post viral lysis.

Virus mediated cancer cell lysis makes tumor associated antigens available for antigen presenting cells (APC), and an increased T cell mediated immune response has been shown to accommodate oncolytic virus therapy. (Gujar S A et al., Mol Cancer Ther., November; 9(11):2924-33 (2010)).

e. Cells

In some embodiments, the additional therapeutic is cells, for example immune cells. For example, a subject's own lymphocytes can be used as an immunotherapy. Cells can be removed from a subject and the lymphocytes can be isolated using techniques commonly known in the art. Activating factors can be added to the isolated lymphocytes. The activating factors can be anything that activate, stimulate or cause proliferation of the lymphocytes. For example, cytokines such as IL-2 or IL-15 can be used as an activating factor (Klebanoff et al., PNAS, 101:1969-74, 2004). After culturing the isolated lymphocytes with one or more activating factors, the activated lymphocytes are returned to the subject.

In one embodiment, T cells specific for a particular tumor antigen can be isolated and proliferated in culture before being re-administered to the subject.

The cells can be from a subject other than the subject being treated, for example heterologous donor. The cells can be stem cells.

Some immunotherapies use antigen presenting cells as a vector. For instance, dendritic cells can be loaded with a therapeutic peptide using in vitro or ex vivo techniques. The therapeutic peptide loaded dendritic cells can then be administered to a patient. Sipuleucel-T, which can be used to treat prostate cancer, was the first FDA approved therapeutic cancer vaccine (Cheever and Hignao, Clin. Cancer Res., 17:3520-3526 (2011). A patient's dendritic cells can be isolated and activated. The activation occurs by pulsing the dendritic cells with an antigen, for example a prostate tumor antigen such as prostatic acid phosphatase (PAP). The cells are readministered to the patient and act as antigen presenting cells which specifically present the antigen via MHC I molecules to CD8+ cells. The CD8+ cells can recognize the MHC I peptide complex and become activated. The activated or cytotoxic T cells can then target and destroy the patient's prostate tumors. Lapuleucel-T uses Her2/neu as the pulsing peptide and is a treatment for breast cancer (Peethambaram et al., Clin Cancer Res., 15:5937-44, (2009)).

2. Chemotherapy

In some embodiments, the second therapeutic is a chemotherapeutic agent. Chemotherapeutics include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present methods contemplate any chemotherapeutic agent that may be employed or known in the art for treating or preventing cancers.

3. Radiotherapy

In some embodiments the second therapeutic is radiation therapy (also referred to as radiation oncology, or radiotherapy, and sometimes abbreviated to XRT or DXT). Radiation therapy is the medical use of radiation, typically ionizing radiation and includes, but is not limited to, external beam radiation, brachytherapy, and radioisotope therapy. Radiation therapy can be used as part of cancer treatment regime to control or kill malignant cells.

### Examples

“PTD-TFAM”, “TFAM”, and “rhTFAM” as used in examples below is a fusion protein with a protein transduction domain, a mitochondrial localization signal, and a TFAM polypeptide.

### Example I

### rhTFAM Increases Classical MHC I Expression on Tumor Cells

Methods

A pancreatic carcinoma cell line, Mia PaCa 2, and a gliobastoma cell line, U-87, were utilized in xenograft experiments. Eight weeks old male nude mice were maintained under sterile condition and injected subcutaneously with either the Glioblastoma Multiforme (GBM) cell line U-87 or the pancreatic cancer line cell Mia PaCa2 (5×10⁶) suspended in 100 μl of matrigel into the right flank of immunosuppressed (nude) mouse strain. When the tumor size reached approximately 100 mm³ volume mice were grouped into groups of eight mice each and started dosing. The mice were treated every four days with vehicle (50 sorbitol 2×PBS), or 0.33, 0.5, 0.66 or 1.0 mg/kg of rhTFAM. The control was vehicle which consisted of Upon termination of the experiment (day 32) the tumors were excised and analyzed by western blot analysis. Western blots were performed to determine the presence and amount of MHC I expression.

Results

An increase in MHC I expression was detected in the pancreatic carcinoma cell line in the presence of rhTFAM. FIG. 1 is a bar graph showing the results of western blot normalized to vehicle control (100%). The presence of rhTFAM provided an increase of MHC I of over 100% compared to controls, i.e., the absence of rhTFAM.

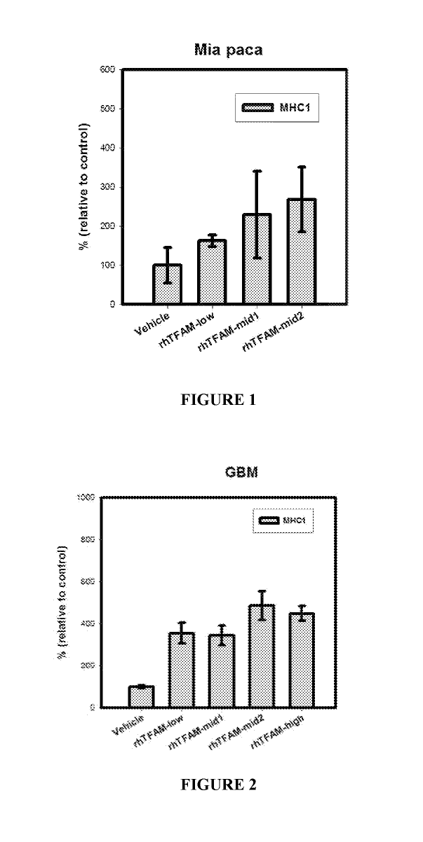

An increase in MHC I expression was detected in Glioblastoma cells in the presence of rhTFAM. FIG. 2 is a bar graph showing the results of western blot normalized to vehicle control (100%). The presence of rhTFAM provided an increase of MHC I of over 100% compared to controls, i.e., the absence of rhTFAM.

Both pancreatic and glioblastoma cell lines showed a statistically significant increase in MHC I expression in the presence of rhTFAM.

### Example II

### rhTFAM Increases HLA-G Expression

Methods

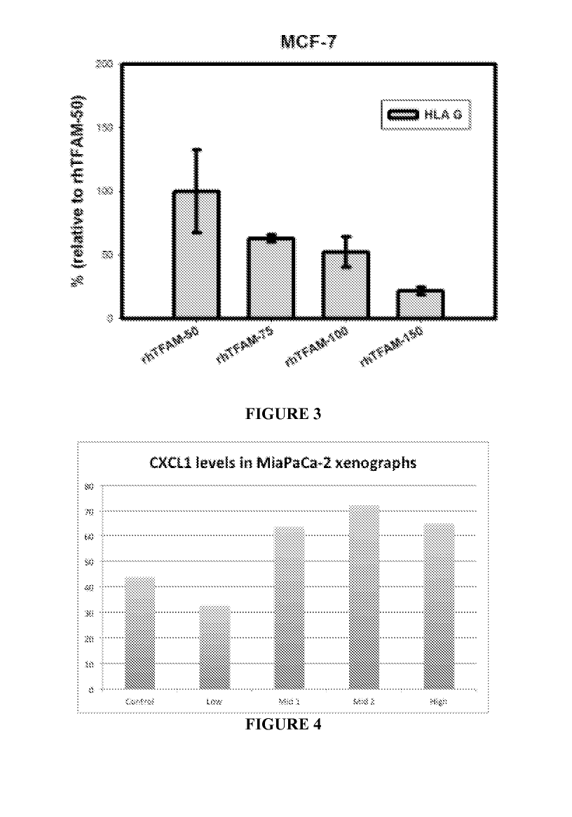

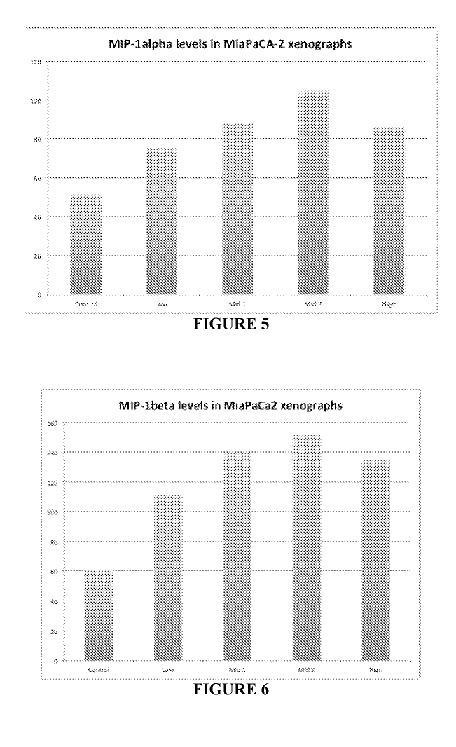

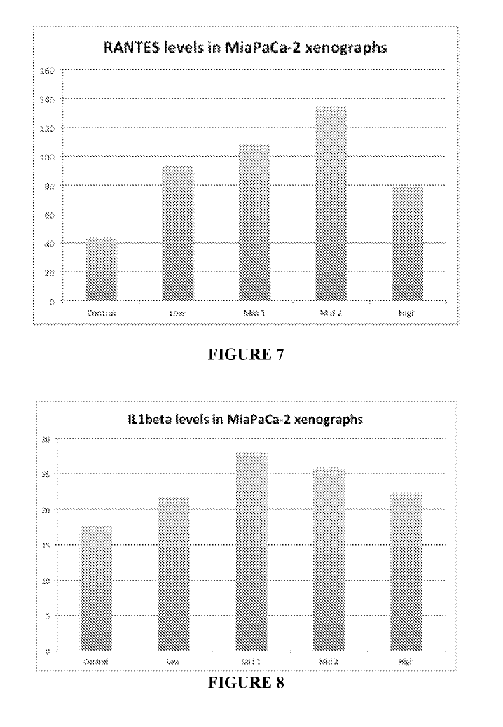

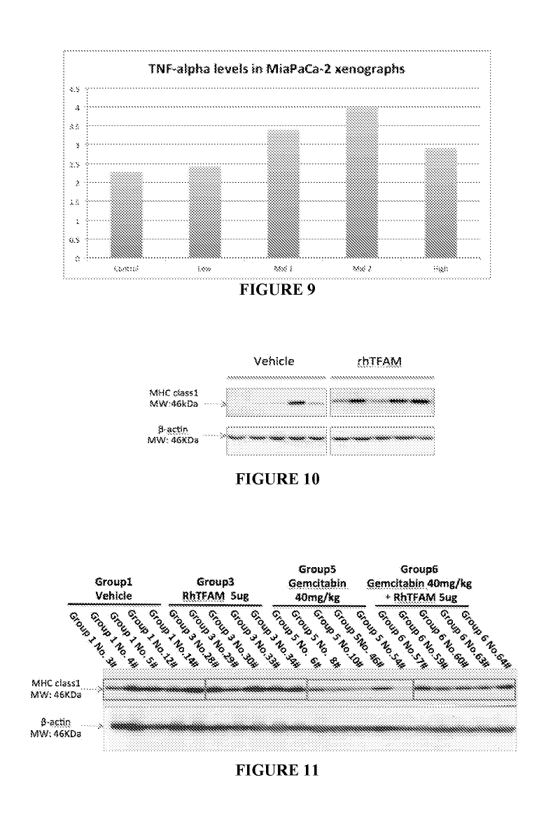

Eight weeks old female nude mice were maintained under sterile condition and injected subcutaneously with MCF-7 cell (5×10⁶) suspended in 100 μl of ice-cold matrigel into the right flank of the mice. Seventeen β-estradiol pellets were implanted subcutaneously around the left forearm using a trochar. When tumor size was approximately 100 mm³, mice were grouped into five groups of four mice each and dosing was started with rhTFAM or vehicle. The test article was administered in four different amounts, namely 0.333, 0.5, 0.666, 1.0 mg/kg of rhTFAM (or 50, 75, 100 and 150 μl injected volume as indicated in FIG. 3). Upon termination of the experiment (day 56), the tumors were excised and analyzed by western blot analysis. No tumors were excised from the vehicle group, as none were alive at the termination of the study. Western blots were performed to determine the presence and amount of HLA-G expression.

Results

HLA-G decreased as concentrations of rhTFAM were increased. HLA-G decreased almost 50% in the presence of rhTFAM-75 and rhTFAM-100 when compared to rhTFAM-50. FIG. 3 is a bar graph showing the results of western blot normalized to rhTFAM-50 (100%).

### Example III

### rhTFAM Increases Cytokine Levels in Tumor Xenographs

Methods

After seven days of acclimatization, 40 nude mice were injected subcutaneously with MiaPaCa2 (5×10⁶) suspended in 100 μl matrigel into their right flank. When tumor size was approximately 100 mm³, mice were grouped into five groups of eight mice each and started dosing with vehicle or rhTFAM by tail vein injection every four days. Tumor sizes were measured twice a week for a total of 60 days.

Tumor samples were frozen and at a later time point subjected to measurements of cytokine and chemokine levels utilizing a Milliplex Map Kit for human cytokines/chemokines, magnetic bead panel (Millipore Corp.), which was utilized according to manufacturer's instructions.

Results

All concentrations of rhTFAM significantly reduced tumor growth. Numerous cytokines were up regulated in MiaPaCa-2 xenographs upon TFAM treatment, an effect that was in most cases dose dependent. Nude mice do not have T cell lymphocytes. It is noted that increases in a number of macrophage-expressed chemokines were detected.

### Example IV

### rhTFAM Increases MHC I Expression in GBM Cell Line Ln229 Inoculated In Situ

Materials and Methods

Cells, Mice and Xenographs

LN229 human glioblastoma cell line was grown as monolayer culture in RPMI-1640 medium supplemented with 10% heat inactivated fetal calf serum, 100 U/ml penicillin and 100 g/ml streptomycin at 37° C. in an atmosphere of 5% CO₂ in air. The tumor cells were routinely subcultured twice weekly by trypsin-EDTA treatment. The cells growing in an exponential growth phase were harvested and counted for tumor inoculation. Each mouse was intracranially injected with 1×10⁵ LN-229 cells suspended in 2 μl phosphate-buffered saline (PBS) over 5 min, 2.5 mm intraparenchymally, 2 mm lateral from and 0.5 mm anterior to the bregma (Day 0). The treatments were started at 7 days post tumor cell inoculation (Day 7). The day was denoted as PG-D1 the animals were treated according to Table 2. Vehicle treated animals were treated with PBS.

TABLE 2
Treatment Schedule for LN229 Xengraph Assay

| | | | | Dosing | Dosing | |
|---|---|---|---|---|---|---|
| Groups | | Treatment | Dose | Volume | Route | Schedule |
| 1 | 0 | Vehicle | — | 100 uL | i.v. | Once every 4 days |
| 2 | 0 | rhTFAM | 1 ug (Low) | 100 ul | i.v. | Once every 4 days |
| 3 | 0 | rhTFAM | 5 ug (Middle) | 100 ul | i.v. | Once every 4 days |
| 4 | 0 | rhTFAM | 10 ug (High) | 100 ul | i.v. | Once every 4 days |
| 5 | 0 | Temozolomide | 10 mg/kg | 10 ml/kg | p.o. | QD × 5/week × 2 |
| | | | | | | weeks |
| 6 | 0 | Temozolomide + | 10 mg/kg + | 10 ml/kg + | p.o. + | QD × 5/week × 2 |
| | | rhTFAM | 5 ug (Middle) | 100 ul | i.v. | weeks + Once every |
| | | | | | | 4 days |

Western Blots

Tumors were collected and cut into 2 halves at termination. One half was snap frozen. Western blotting (MHC Class I) was performed using the lysates from frozen tumor samples. Western Blot conditions: Lysis Buffer: RIPA buffer; Total protein: 20 μg; Primary antibody: anti-MHC 1 (Santa Cruz mouse monoclonal antibody at 1:500 (#sc-55582)); Secondary antibody: goat anti-mouse at 1:5000); Exposure time: 5 min.

Results

As shown in FIG. 10, MHC I expression is detectible in all five TFAM treated tumors, and show stronger overall expression than in the untreated (vehicle) control.

### Example V

### PTD-TFAM Reduces the Decrease of MHC-I Expression in Gemcitabine Treated Mice in an Orthotopic Model of Pancreatic Cancer

Materials and Methods

Cells, Mice and Xenographs

MiaPaCa-2 tumor cells was maintained in vitro as monolayer culture in DMEM medium supplemented with 10% fetal bovine serum, 2.5% horse serum, 100 Um′ penicillin and 100 μg/ml streptomycin, and 2 mM L-glutamine at 37° C. in an atmosphere of 5% CO₂ in air. The tumor cells were routinely subcultured twice weekly by trypsin-EDTA treatment. Cells in an exponential growth phase will be harvested and counted for tumor inoculation. Before orthotopic implantation, 5×10⁶ MiaPaca-2 cells in 50 μl PBS mixed with 50 μl Matrigel were inoculated subcutaneously at the right flank of each BALB/c nude mouse. Upon subcutaneous tumor size reaching about 500 mm³, tumors were collected for orthotopic implantation.

For orthotopic MiaPaca-2 xenografts, mice were anesthetized by intraperitoneal injection of pentobarbital sodium (50 mg/kg) before tumor inoculation. The abdominal skin was sterilized and laparotomy was performed to expose the pancreas. Each mouse was inoculated with a subcutaneous MiaPaca-2 tumor fragment (2-3 mm in diameter) in the subcapsular region of the pancreas for tumor development. The abdominal wall was closed using No. 6 suture and then sterilized with povidone iodine solution. The day of surgery was denoted as Day 0 (D0). The treatments with vehicle, gemcitabine only, PTD-TFAM only, and PTD-TFAM in combination with gemcitabine were started at 10 days post tumor fragment inoculation. The day was denoted as Day 1 of treatment (PG-D1). PTD-TFAM was administered at a dose of 0.15 mg/kg every fourth day commencing at day PG-D1. Gemcitabine was administrated at a dose of 40 mg/kg bodyweight every 3 days, starting at day PG-D1.

Western Blots

Tumors were collected and cut into 2 halves at termination. One half was snap frozen. Western blotting (MHC Class I) was performed using the lysates from frozen tumor samples. Western Blot conditions: Lysis Buffer: RIPA buffer; Total protein: 25 μg; Primary antibody: anti-MHC 1 (Santa Cruz mouse monoclonal antibody at 1:500 (#sc-55582)); Secondary antibody: goat anti-mouse at 1:5000); Exposure time: 1 min.

Materials and Methods

As shown in FIG. 11, MHC I expression is decreased in Gemcitabine treated samples, a decrease that is mitigated by rhTFAM treatment.

1. A method for inducing, enhancing, or promoting an immune response in a subject comprising administering to the subject an effective amount of transcription factor A-mitochondrial (TFAM) to increase expression of Major Histocompatibility Complex I (MHC I or reduce the expression of HLA-G on the surface of cells or both.

2. (canceled)

3. A method for inducing, enhancing, or promoting an immune response in a subject comprising administering to the subject an effective amount of a transcription factor A-mitochondrial (TFAM) to increase expression of one or more cytokines or chemokines.

4. The method of claim 1 wherein the TFAM is a fusion protein.

5. The method of claim 1 wherein the TFAM induces, enhances, or promotes an immune response to an antigen.

6. The method of claim 1 wherein the antigen is a tumor antigen.

7. The method of claim 1 wherein the immune response is activation of cytotoxic T cells to kill cells displaying the antigen.

8. A method of treating cancer comprising administering to a subject in need thereof, an effective amount of transcription factor A-mitochondrial (TFAM) to induce, enhance, or promote an immune response against cancer cells.

9. The method of claim 8 wherein the TFAM is administered in an effective amount to (a) increase expression of HLA-A, HLA-B, or HLA-C on the surface of cancer cells, (b) decrease expression of HLA-G on the surface of cancer cells, or (c) combinations thereof.

10. The method of claim 1 wherein the TFAM is expressed in an effective amount to increase expression of one or more cytokines from cells in or around the microenvironment of the cancer cells.

11. The method of claim 1 wherein the immune response is activation of cytotoxic T cells against the cancer cells.

12. The method of claim 1 wherein the TFAM is a fusion protein.

13. The method of claim 1 wherein the fusion protein comprises (i) a protein transduction domain, (ii) a mitochondrial localization signal, and (iii) TFAM polypeptide.

14. The method of claim 13 wherein the protein transduction domain comprises 7-15 arginine residues.

15. The method of claim 13 wherein the mitochondrial localization signal comprises the mitochondrial localization signal of superoxide dismutase 2 (SOD2).

16. The method of claim 13 wherein the TFAM polypeptide is a mature TFAM polypeptide.

17. The method of claim 13 wherein the fusion protein comprises the amino acid sequence of SEQ ID NO:26.

18. The method of claim 1 further comprising administering to the subject a second therapeutic agent.

19. The method of claim 18 wherein the second therapeutic agent is a vaccine.

20. The method of claim 19 where the vaccine comprises a peptide antigen, a fusion protein antigen, or a vector expressing the peptide or the fusion protein antigen.

21.-35. (canceled)
